(19) United States
(12) Patent Application Publication
(10) Pub. No.: US 2015/0018715 A1
(43) Pub. Date: Jan. 15, 2015

(54) Devices and Methods to Reduce Contamination of Fluid Collected from a Patient

(71) Applicant: Juan Nepomuc Walterspiel, Belmont, CA (US)

(72) Inventor: Juan Nepomuc Walterspiel, Belmont, CA (US)

(21) Appl. No.: 14/502,208

(22) Filed: Sep. 30, 2014

Related U.S. Application Data
(63) Continuation of application No. PCT/US2014/021223, filed on Mar. 6, 2014.
(60) Provisional application No. 61/832,659, filed on Jun. 7, 2013.

(51) Int. Cl. B01L 3/00; A61B 10/00; A61B 5/154

(52) U.S. Cl. CPC B01L 3/565; A61B 5/154; A61B 10/0045
USPC 600/576; 422/570

(57) ABSTRACT

The invention includes devices and methods for obtaining samples of blood or other bodily fluids with reduced levels of contamination. Fluid obtained from a subject may be contaminated by skin cells, bacteria, fungi, viruses, phages, their respective RNA, DNA, and/or other undesirable molecules, or disinfectants. A first amount of fluid is injected from the subject through an distal needle and a proximal needle penetrates a first portion of a device having a sequestration chamber with sub-atmospheric pressure therein. A first portion of the fluid, containing contaminants, is deposited into the sequestration chamber. The proximal needle is then moved through a second portion of the sequestration chamber and into a collection container. Because contaminants are removed from the sample, analysis and diagnosis of a subject's condition becomes more reliable and accurate. Additional devices and methods can be used to obtain relatively uncontaminated samples from cell culturing vessels.

### CLAIM OF PRIORITY

This application claims priority to U.S. Provisional Patent Application No. 61/832,659, filed Mar. 19, 2013 entitled “Device to Sequester First Amount of Blood Drawn into a Vacuum Container,” Juan Nepomuc Walterspiel, Inventor. This application is incorporated herein fully by reference.

### FIELD OF THE INVENTION

This invention relates to devices and methods for obtaining samples of blood or other fluids. More particularly, this invention relates to devices and methods for obtaining samples of blood or other fluids with reduced contamination. Even more particularly, this invention relates to devices and methods for obtaining samples of blood or other fluids in which a first portion of the sample may be contaminated, and the remainder of the sample, being relatively uncontaminated, is collected.

### BACKGROUND

Analysis and processing of samples of biological fluids is an important aspect of diagnosis and evaluation of many disorders and diseases. Generally, a sample of blood or other body fluid is obtained by inserting a needle or similar device into a blood vessel, infected lesion, malignancy or suspected pathological fluid accumulation and withdrawing a sample into a container. Sample containers are often used to collect and store samples, and such containers are in wide use worldwide.

### SUMMARY

Blood culture contamination represents an ongoing source of frustration for clinicians and hurts patients. The median adult in-patient contamination rate is 2½ percent and can range from 0.6% to 6%. The estimated additional cost for unnecessary treatments in adults and hospital admissions in children is around $1,000. A device cost of $25 would break even and save valuable antibiotics. Any price below $20 would save costs and valuable antibiotics.

Interferon stimulation tests to diagnose tuberculosis are performed in 12,000,000 health workers each year in the United States. The results can switch from positive to negative and vice versa. This is thought to be due to contamination with skin organisms. The exact cost from prophylactic treatment and side effects (at least 6 months) is unknown, but is considered to be significant.

I have identified a problem in the field, namely, that in using conventional devices and methods for obtaining a sample of blood or other fluid, a portion of the skin and with it, microorganisms may be inadvertently obtained as well. The microorganisms vary by location and may include numerous undesired components, including bacteria, yeasts, fungi, viruses, phages in either single, mixed, aggregated, or biofilm form and their components. When such undesired components are obtained and mixed within a container, such as a vacuum tube or other sample container, the contaminants can compromise analysis of the blood or other fluid sample, producing unreliable results.

I have therefore developed new devices and methods to overcome this problem. In general, the devices and methods of this invention include a device having a sidewalls, a first end, and a second end defining a sequestration chamber therein. The device may be a cap or other device that can be used in conjunction with a vacuum tube or other sample collection container. Additionally, a device may be connected to an inflow tube and an outflow tube, so that fluid can be transported from one location to another, with the device disposed therebetween. Improved devices include a sequestration chamber or space within the device that is sealed from the atmosphere and has a sub-atmospheric pressure within it. The sequestration chamber can have two portions that are penetrable by a collection device having a sample collection needle, generally one at the upstream side of the space (an “upstream needle” or “distal needle”), and another needle at the downstream side of the space (a “downstream needle” or “proximal needle”).

To obtain a sample of biological fluid, a first upstream or distal needle is generally inserted into a subject's vein or other fluid-containing space. The distal needle may be attached via a tube to a second, downstream or proximal needle that can then be inserted into the device. The two needles may be connected by tube to permit fluid to flow from the first to the second needle. When a sample of fluid is being obtained from a patient using the distal needle, the proximal needle can be inserted into the sequestration chamber within the device, and sub-atmospheric pressure within the sequestration chamber acts to draw a first portion of the sample into the sequestration chamber. Generally, the first portion of the sample contains the undesirable contaminants. When the proximal needle passes out of the sequestration chamber in the device into another container (a sample container or “collection container”), the contaminants remain in the sequestration chamber of the device to sequester a first amount of fluid containing contaminants. The remainder of the sample can then be deposited into the collection container and be relatively free of contaminants. Similar devices can be adapted to be used to collect relatively uncontaminated samples from cell or tissues culture vessels.

In other aspects, this invention includes a second sequestration chamber. Thus, after a first portion of a sample has been deposited in a first sequestration chamber, the proximal needle may be inserted into a second sequestration chamber to deposit a second sample, that may contain some contaminants. By using two sequestration chambers in series, further reduction of contamination can be achieved. It can be appreciated that a third, fourth, or more sequestration chambers can be used to further reduce contaminants introduced into a collection container.

Analysis of the relatively uncontaminated sample can be performed with increased accuracy and reliability, thereby permitting accurate diagnosis and analysis of a patient's condition, thereby improving health care.

### DESCRIPTION OF EMBODIMENTS

### Embodiment 1

A device, comprising: a stopper having a bottom portion, a sidewall portion, and a top portion defining a sequestration chamber impermeable to air, having sub-atmospheric pressure therein; said top and bottom portions being penetrable by a needle.

### Embodiment 2

The device of embodiment 1, further comprising a sample container having a closed end and an open end, said sample container sized to sealingly accept said stopper.

### Embodiment 3

The device of embodiment 1, said stopper comprising: a screw cap having a bottom portion, a sidewall portion, and a top portion defining a sequestration chamber impermeable to air, said sequestration chamber having sub-atmospheric pressure therein, said screw cap threadably engaged with corresponding threads on said sample container, said screw cap penetrable by a needle; and a sample collection container having a closed end and an open end, said sample collection container sized to threadably and sealingly engage said screw cap.

### Embodiment 4

The device of any of embodiments 1 to 3, where said sequestration chamber has a volume ranging from about 1 cubic millimeter to about 10,000 cubic millimeters.

### Embodiment 5

The device of any of embodiments 1 to 4, further comprising one or more additional sequestration chambers containing sub-atmospheric pressure.

### Embodiment 6

The device of any of embodiments 1 to 6, where said sample container has sub-atmospheric pressure therein.

### Embodiment 7

The device of any of embodiments 1 to 6, where the pressure within at least one of said sequestration chambers is in the range of about 1% to about 90% of the surrounding atmospheric pressure.

### Embodiment 8

The device of any of embodiments 1 to 7, at least one of said walls being transparent.

### Embodiment 9

The device of embodiment 5, said sequestration chambers varying in progressive fashion from larger to smaller in a distal direction.

### Embodiment 10

The device of embodiments 5, said sequestration chambers varying in progressive fashion from smaller to larger in a distal direction.

### Embodiment 11

The device of any of embodiments 1 to 10 said sequestration chamber sized to hold a volume of fluid from about 1 cubic millimeter to about 10,000 cubic millimeters.

### Embodiment 12

The device of any of embodiments 1 to 11, said sequestration chamber comprising a window to permit an observer to see within the sequestration chamber or sequestration chambers.

### Embodiment 13

The device of any of embodiments 1 to 12, where a top portion of the stopper comprises a flange extending above the top of an open end of the sample collection container.

### Embodiment 14

The device of any of embodiments 1 to 13, where the bottom portion of at least one sequestration chamber has an opening extending from the sequestration chamber through the bottom portion of the stopper or screw cap, the opening further containing a porous, absorbent material that when wetted by a fluid, fills said opening, and becomes impermeable to gas or fluid.

### Embodiment 15

The device of embodiment 14, where said absorbent material is selected from the group consisting of packed or woven fibers of cotton, cellulose, cellulose fibers, polyamines, cationic starch, rayon, cotton, silk, nylon, microporous polyvinylidene difluoride (PVDF), microporous mixed cellulose esters (MCE), and poly tetra fluoro ethylene (PTFE), mixed cellulose esters (MCE), acrylonitrile butadiene styrene (ABS), butadiene styrene rubber (BS), cyclohexanedimethanol (CHDM), cellulose nitrate-cellulose acetate (CN-CA), ethylene propylene terpolymer rubber (EPDM), ethylene vinyl acetate (EVA), high density polyethylene (HDPE), high density polypropylene (HDPP), high impact polystyrene (HIPS), low density polyethylene (LDPE), methyl methacrylate ABS (MABS), nitrile butadiene rubber (NBR), neoprene (NPRN), polyamide (PA), polybutylene terephthalate (PBT), polycarbonate (PC), polyethylene (PE), polyethersulfone (PES), polyethylene terephthalate (PET), PET modified with glycolor (PETG), polyimide (PI), butyl rubber (PIB), polyoxymethylene (POM), polypropylene (PP), polystyrene (PS), polysulfone (PSU), polytetrafluoroethylene (PTFE), polyurethane (PU), polyvinyl acetate (PVA), polyvinyl chloride (PVC), styrene butadiene (SB), styrene butadiene rubber (SBR), stainless steel (SS), thermoplastic elastomer (TPE), thermoplastic rubber (TPR).

### Embodiment 16

The device of embodiment 15, where said absorbent material has an effective pore size ranging from 0.02 μm to 1 mm.

### Embodiment 17

The device of any of embodiments 1 to 16, said stopper having a sidewall penetrable by a needle inserted through the top portion and through the bottom portion without penetrating into the sequestration chamber.

### Embodiment 18

A device for collecting a sample of fluid from a culture container, comprising:

a first side, a second side, a third side, a fourth side, a top, and a bottom, defining a first sequestration chamber therebetween, said first sequestration chamber having sub-atmospheric pressure therein, said top and bottom being penetrable by a needle.

### Embodiment 19

The device of embodiment 18, further comprising a second sequestration chamber having sub-atmospheric pressure therein, and separated from said first sequestration chamber by the bottom of said first sequestration chamber.

### Embodiment 20

A method for collecting a sample of fluid from a subject, comprising;

a. providing a device of any of embodiments 1 to 18;

b. providing a sampling device having a distal needle and a proximal needle connected to each other by a tube defining a continuous fluid passageway;

c. inserting the distal needle into a fluid-filled cavity of a subject, permitting said fluid to flow through said distal needle, said tube, and into the proximal needle;

d. inserting the proximal needle through the top portion of said stopper and into said sequestration chamber, permitting a first portion of the fluid to be drawn into the sequestration chamber; then

e. inserting the proximal needle through said bottom portion of said sequestration chamber and into a sample container, thereby permitting a second portion of fluid to flow into the sample container.

### Embodiment 21

The method of embodiment 20, where said fluid filled cavity of a subject is a fluid-filled vessel or aspirable lesion.

### Embodiment 22

The method of embodiments 20, said fluid being blood, pus, lymph, cerebrospinal fluid, exudate, transudate, or fluid from an infected lesion.

### Embodiment 23

The method of embodiments 20 to 22, comprising:

retrieving a portion of the second portion of fluid from the sample container without contamination by the first portion of the fluid.

### Embodiment 24

A method for obtaining a sample of culture fluid from a culture vessel, comprising:

a. providing a culture vessel containing culture medium;

b. providing a sampling device having a distal needle and a proximal needle connected to each other by a tube defining a continuous fluid passageway therebetween;

c. providing a device of embodiment 16;

d. inserting said distal needle into said culture vessel;

e. permitting a first sample of culture fluid to be drawn into a first sequestration chamber of said device; then

f. inserting said proximal needle through the bottom of said first sequestration chamber; and

g. permitting said culture fluid to flow through said proximal needle into a culture collection vessel.

### Embodiment 25

A method for obtaining a sample of culture fluid from a culture vessel, comprising:

a. providing a culture vessel containing culture medium;

b. providing a sampling device having a distal needle and a proximal needle connected to each other by a tube defining a continuous fluid passageway therebetween;

c. providing a device of embodiment 17;

d. inserting said distal needle into said culture vessel;

e. permitting a first sample of culture fluid to be drawn into said first sequestration chamber of said device; then

f. inserting said proximal needle through the bottom of said first sequestration chamber and into said second sequestration chamber; thereby permitting said culture fluid to flow into a second sequestration chamber; then

g. inserting said proximal needle through the bottom of said second sequestration chamber, there by permitting culture fluid to flow out of said proximal needle into a culture collection vessel.

### BRIEF DESCRIPTION OF THE FIGURES

This invention is being described with reference to specific embodiments thereof. Additional aspects of this invention can be understood with reference to the figures, in which:

FIG. 1A depicts a side view schematic drawing of an embodiment 100 of a stopper of the invention, said stopper containing a sequestration chamber 120 at sub-atmospheric pressure, said stopper containing top 105, bottom 106, sidewalls 115 and 116, each of which are penetrable by a tip end of the proximal needle.

FIG. 1B depicts a side view schematic drawing of an embodiment 101 of the invention, said stopper inserted into a sample container 150, creating a sealed sequestration chamber 155 with sub-atmospheric pressure therein, said stopper penetrated by tip end 140 of proximal needle 135.

FIG. 1C depicts a side view schematic drawing of an embodiment 102 of the invention, with stopper penetrated by the tip end 140 of proximal needle 135, said needle being inserted through the stopper sequestration chamber 120 and into collection container 155.

FIG. 1D depicts a side view schematic drawing of an embodiment 103 of the invention, with stopper penetrated by the tip end 141 of proximal needle 136, said needle being inserted into the stopper without making contact with sequestration chamber 120 within said stopper and into collection container 155.

FIG. 1E depicts a top view schematic drawing of an embodiment 104a of this invention, depicting a transparent side window 125 in said stopper, through which an operator or phlebotomist can observe blood or fluid enter sequestration chamber 120.

FIG. 1F depicts a top view schematic drawing of an embodiment 104b of the invention, depicting a gap in said stopper, through which an operator or phlebotomist can observe blood or fluid enter sequestration chamber 120.

FIG. 1G depicts a top view schematic drawing of an embodiment 104c of the invention, depicting a gap in said stopper being adjacent to container 150 through which an operator or phlebotomist can observe blood or fluid enter sequestration chamber 120.

FIG. 2A depicts a side view schematic drawing of an embodiment 200 of a stopper of the invention, said stopper inserted into container 250, said stopper 200 containing a sequestration chamber 220 having sub-atmospheric pressure therein, said stopper connected to sequestration chamber 255 through opening 230, said opening containing wettable occlusion material 231.

FIG. 2B depicts a side view schematic drawing of an embodiment 201 of the invention as shown in FIG. 2A, with the stopper penetrated by tip end 240 of proximal needle 235, said needle being inserted into the stopper into sequestration chamber 220.

FIG. 2C depicts a side view schematic drawing of an embodiment 202 of the invention as shown in FIG. 2A, showing the stopper, opening 230, with absorbent occluding material 231 of FIG. 2B now becoming wetted, producing occlusion material 231a, which occludes opening 230. With occlusion of opening 230, neither fluid nor gas can pass from sequestration chamber 220 into the space within collection container 255. Tip end 240 of proximal needle 235, is shown inserted into the stopper making contact with sequestration chamber 220 and passing through sequestration chamber 220 and into collection container 255 to enable the drawing and storing of a test sample of blood or other bodily fluid with reduced levels of contamination.

FIG. 2D depicts a side view schematic drawing of an embodiment 203 of this invention as shown in FIG. 2C, and sampling needle 236 within sample container 255 to enable the drawing and storing of a test sample of blood or other bodily fluid, with reduced levels of contamination.

FIG. 3A depicts a side view schematic drawing of an embodiment 300 of a stopper of the invention, said stopper inserted into a container 350, said insertion creating a sealed sequestration chamber 355 having sub-atmospheric pressure therein, said stopper 300 containing stacked sequestration chambers 320 and 321 at sub-atmospheric pressure.

FIG. 3B depicts a side view schematic drawing of an embodiment 301 of a stopper as shown in FIG. 3A, with said stopper penetrated by tip end 340 of proximal needle 335, said needle being inserted into the stopper through sequestration chamber 320 and into sequestration chamber 321.

FIG. 3C depicts a schematic drawing of an embodiment 302 of a stopper as shown in FIG. 3A, with said stopper penetrated by tip end 340 of proximal needle 335, said needle shown inserted through stacked stopper sequestration chambers 320 and 321 and into collection container 355.

FIG. 3D depicts a schematic drawing of an embodiment 303 of a stopper as shown in FIG. 3A, with said stopper 303 penetrated by tip end 341 of a sampling needle 336, said needle being inserted into the stopper without making contact with sequestration chambers 320 or 321.

FIG. 4A depicts a side view schematic drawing of an embodiment 400 of a screw cap stopper of the invention, with threads and groves 460, said screw cap threadably engaged to container 450 having sub-atmospheric pressure therein, and creating a sealed sequestration chamber 455 having sub-atmospheric pressure therein, said screw cap stopper 400 containing a sequestration chamber 420 at sub-atmospheric pressure, penetrated by tip end 440 of proximal needle 435.

FIG. 4B depicts a side view schematic drawing of an embodiment 401 of a screw cap stopper of the invention as seen in FIG. 4A, said screw cap stopper 400 penetrated by tip end 441 of proximal needle 436, said needle making contact and passing through stopper sequestration chamber 420 and entering into sequestration chamber 455 to enable the drawing and storing of a test sample of blood or other bodily fluid, with reduced levels of Contamination.

FIG. 4C depicts a side view schematic drawing of an embodiment 402 of a screw cap stopper of the invention as depicted in FIG. 4A, showing screw cap top 405 penetrated by proximal needle 436, showing tip end 441 within collection container 455, said needle being inserted into the stopper without making contact with sequestration chamber 420 to enable the drawing and storing of a test sample of blood or other bodily fluid, with reduced levels of contamination.

FIG. 5A depicts a side view schematic drawing of an embodiment 500 of a screw cap stopper of the invention, said screw cap stopper threadably attached to container 550 creating a sealed sequestration chamber 555 at sub-atmospheric pressure, said screw top stopper containing sequestration chamber 520, said screw top stopper connected to sequestration chamber 555 through opening 530, with contains an absorbent occlusion-creating material 531.

FIG. 5B depicts a side view schematic drawing of an embodiment 501 of with screw cap stopper as shown in FIG. 5A penetrated by tip end 540 of proximal needle 535, said needle being inserted into the screw cap stopper into sequestration chamber 520. FIG. 5B also shows openings 530 containing absorbent, occlusion-creating material 531 therein.

FIG. 5C depicts a side view schematic drawing of an embodiment 502 of the invention as shown in FIG. 5B, said absorbent, occlusion-creating material 531 of FIG. 5B now becoming wetted to form occluding material 531a. By occluding opening 530, gas and fluid cannot pass from sequestration chamber 520 into collection container 555. Tip end 540 of proximal needle 535 is shown through sequestration chamber 520 and into collection container 555 to enable the drawing and storing of a test sample of blood or other bodily fluid, with reduced levels of contamination.

FIG. 5D depicts a side view schematic drawing of an embodiment 503 of the invention with screw cap stopper as shown in FIG. 5B, with the absorbent occluding material 531 of FIG. 5B now becoming wetted thereby forming occlusion material 531a, occluding opening 530, thereby preventing gas or fluid from passing from sequestration chamber 520 into collection container 555. Sampling needle 536 is shown placed through the screw cap stopper without making contact with sequestration chamber 520 within said screw cap stopper and tip 541 is shown within collection container 555 to enable the drawing and storing of a test sample of blood or other bodily fluid, with reduced levels of contamination.

FIG. 6A depicts a side view schematic drawing of an embodiment 600 of a screw cap stopper of the invention. Collection container 650 is shown having sub-atmospheric pressure therein in space 655, said stopper 600 containing stacked, sequestration chambers 620 and 621 each at sub-atmospheric pressure and connected to container 650. Tip end 640 of proximal needle 635 is shown inserted through top 605 of the screw cap stopper into sequestration chamber 620.

FIG. 6B depicts a side view schematic drawing of an embodiment 601 of a screw cap stopper as shown in FIG. 6A penetrated by tip end 640 of proximal needle 635, said needle shown inserted through the stopper, through sequestration chamber 620 and into space 655 of sequestration chamber 621.

FIG. 6C depicts a side view schematic drawing of an embodiment 602 of a screw cap stopper as shown in FIG. 6A showing tip end 640 of proximal needle 635, within space 655 of collection container 650.

FIG. 6D depicts a side view schematic drawing of an embodiment 603 of a screw cap stopper as depicted in FIG. 6A showing tip end 641 of proximal needle 636, said needle shown through the screw cap stopper without making contact with sequestration chambers 620 or 621, said needle being inserted into space 655 of collection container 650 to enable the drawing and storing of a test sample of blood or other bodily fluid, with reduced levels of contamination.

FIG. 7A depicts a side view schematic drawing 700 of an in-line embodiment of the invention, containing a sequestration chamber 720 at sub-atmospheric pressure, penetrated by proximal end 740 of proximal needle 735.

FIG. 7B depicts a side view schematic drawing of in-line embodiment 701 as depicted in FIG. 7A showing tip end 740 of proximal needle 735, said needle being inserted into out flow tube 755 passing through sequestration chamber 720.

FIG. 8A depicts a side view schematic drawing 800 of an in-line embodiment of the invention, said embodiment containing stacked sequestration chambers 820 and 821, each at sub-atmospheric pressure, said embodiment connected to an out flow tube 855.

FIG. 8B depicts a side view schematic drawing of an embodiment 801 similar to that shown in FIG. 8A, where sequestration chamber 820 is penetrated by tip end 840 of proximal needle 835.

FIG. 8C depicts a side view schematic drawing of an in-line embodiment 802 similar to that shown in FIG. 8A, where sequestration chambers 820 and 821 are penetrated by tip end 840 of proximal needle 835.

FIG. 8D depicts a side view schematic drawing of an in-line embodiment 803 similar to that shown in FIG. 8A, where in-line sequestration chambers 820 and 821 are penetrated by tip end 840 of proximal needle 835, said needle penetrating into out flow tube 855.

FIG. 9A depicts a schematic drawing of an in-line embodiment 900 of the invention, containing sequestration chamber 920 having sub-atmospheric pressure therein, said sequestration chamber penetrated by tip end 940 of proximal needle 935, and a distal end 910 of needle 935 said needle drawing from a culture vessel through a puncturable portal 910.

FIG. 9B depicts a schematic drawing of an in-line embodiment 901, similar to that shown in FIG. 9A penetrated by the proximal tip end 940 of needle 935, said needle being inserted into through sequestration chamber 920 and into the out flow tube 955.

FIG. 10 depicts a side view schematic drawing of conventional stopper 1000 of the prior art, having a top portion 1005 and sidewalls 1015 and 1016 made of a resilient material, defining a sequestration chamber between the sidewalls, but without a bottom portion. The stopper is shown inserted into a container 1050 having sub-atmospheric pressure in space 1055 therein.

### DETAILED DESCRIPTION

### Aspects of the Invention

I have identified a problem in the field, namely, that in using conventional devices and methods for obtaining a sample of blood or other fluid, a portion of the skin and with it, microorganisms may be inadvertently obtained as well. The microorganisms vary by location and may include numerous undesired components, including bacteria, yeasts, fungi, viruses, phage in either single, mixed, aggregated, or biofilm form. When such undesired components are obtained and mixed within a container, such as a sample collection container tube or other container, the contaminants can compromise analysis of the blood or other fluid sample, producing unreliable results.

I have therefore developed new devices and methods to overcome this problem. In general, the devices and methods of this invention include a new stopper, cap or other device, that can be used in conjunction with a vacuum tube or other container. Improved devices include a sequestration chamber or space within the device that is sealed from the atmosphere and has a sub-atmospheric pressure within it. The sequestration chamber can have two or more portions that are penetrable by a sample collection needle, generally one at the upstream side of the space, and another at the downstream side of the space, and one at a sidewall.

When a body fluid such as blood or a biopsy or other fluid is collected through a needle or other cutting device, the needle or cutting device has to first transverse the anatomical structures that overlay the blood carrying vessels, or cavities, lesion, fluid accumulations or the site for a biopsy.

In most cases, the first structure to be transected is the skin. Skin is comprised of several layers. The upper keratinized layers are colonized with microorganisms that are not always killed or inactivated by cleaning and sterilization that variably occurs prior to puncture. A needle or cutting device that transverses these structures often carries with it pieces of skin, skin cells and underlying tissues that have been punched out or were scrapped off. The microorganisms, their biofilm structures, and specific molecules attached to or embedded in the skin cells become contaminants that can complicate the analysis and processing of the fluid sample taken.

The presence of such contaminants can lead to erroneous conclusions regarding the presence of a microbial organism in the blood, body fluid, or biopsy material or a false positive evidence for exposure to an infectious agent. An example includes for a blood culture to read false positive with a coagulase negative staphylococcus species. Molecules from contaminants can lead to erroneous conclusions regarding the induction of interferon or cytokines from cells of the immune system. An example includes the false positive finding for past contact with mycobacterium tuberculosis. The respective DNA and RNA sequences from such contaminants can also falsely signal the presence of various organisms, their apparent quantities or their resistance patterns in fluid samples or biopsies, when in fact, they are not present in uncontaminated sample material. These contaminants are generally found in the first portion of blood, body fluid or biopsy material collected.

A way to remedy this is to sequester a first portion of the collected material as a separate portion in parts of the collection device or in a similarly structured in between, and thereby decrease the likelihood that the now sequestered material will contaminate the sample to be analyzed or processed. This disclosure describes structures and functions of devices that accomplish this purpose. Such sequestration can be conveniently accomplished by providing sequestering chambers (or “chambers”) at sub-atmospheric pressure of from about 1% to about 90% of the ambient atmospheric pressure. Such devices can be made in a number of different forms as described herein. However, other devices can be made based on the disclosure and teachings herein.

The sources of fluid are not limited, and may include blood, lymph, peritoneal fluid, cerebrospinal fluid, urine, feces, pus, fluid from an aspirable lesion, including a cyst, bacterial nidus, aqueous humor, vitreous humor, or interstitial fluid.

The types of assays that may be carried out on relatively uncontaminated samples include testing for bacteria, viruses, RNA, DNA, tumor cells, cell viability, presence of secreted molecules including proteins, peptides, amino acids, metabolic products, drugs and their metabolites, and in certain other embodiments, measurement of cell growth rate, and cell death. However, it can be appreciated that any test now being performed on a fluid sample can be improved by the use of devices of this invention, and the principles contained therein.

Devices to Sequester the First Amount of Fluid Drawn into a Collection Container

Certain aspects include a device to obtain test samples of blood and other bodily fluids with reduced levels of contamination.

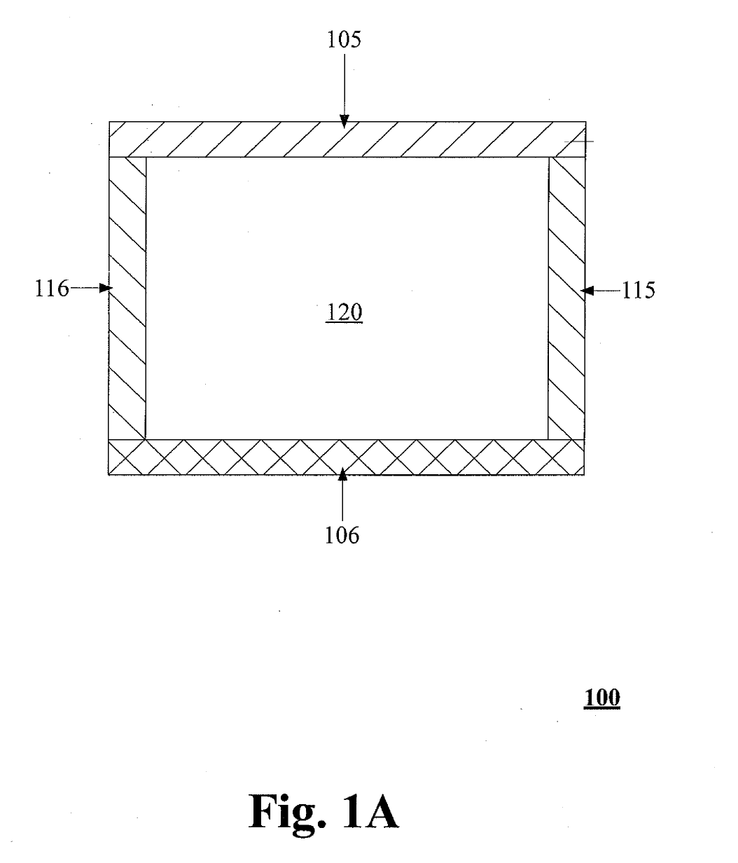

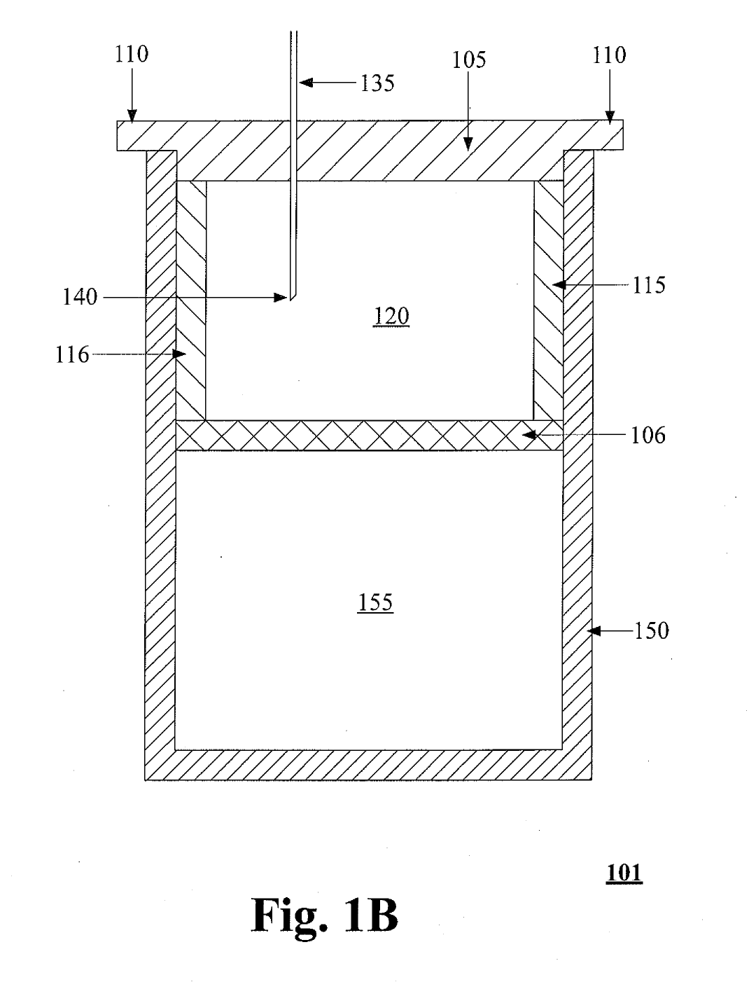

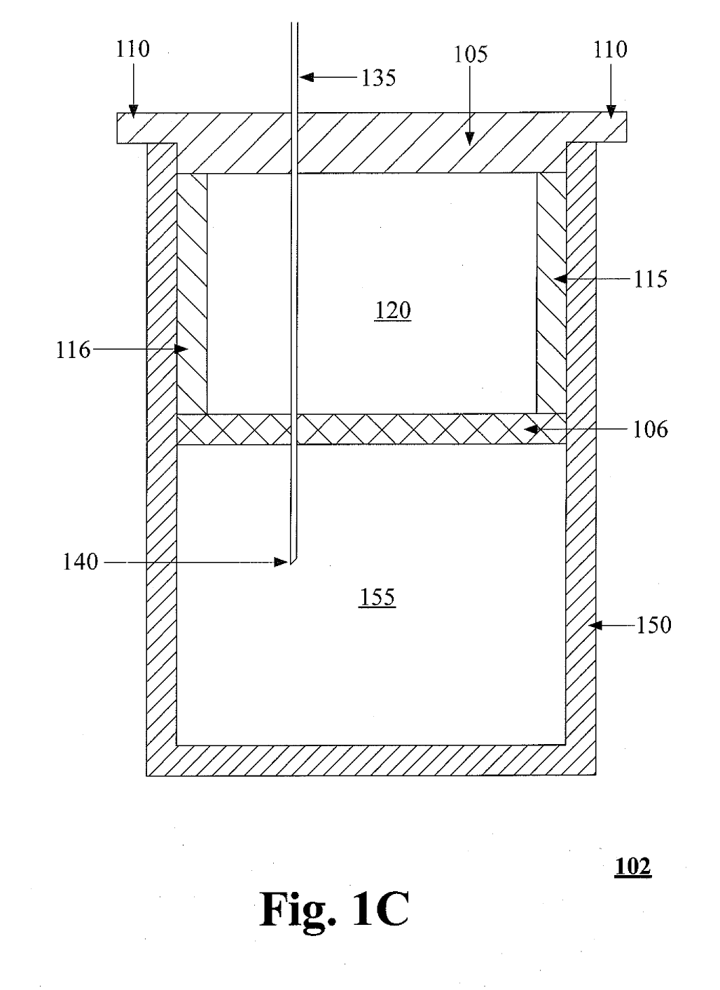

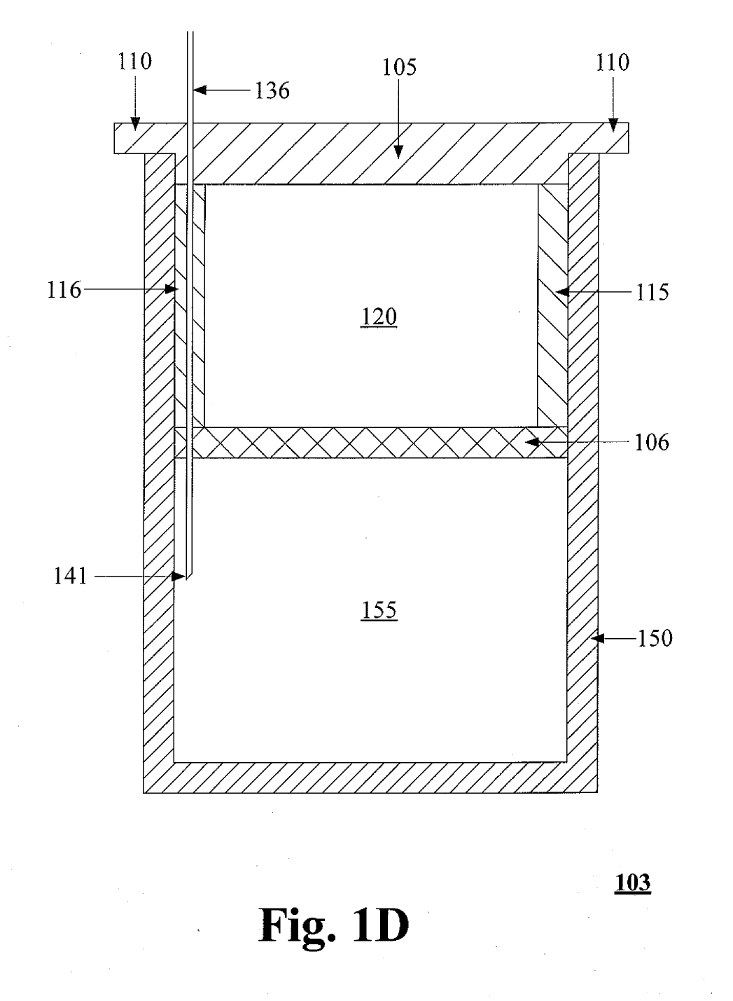

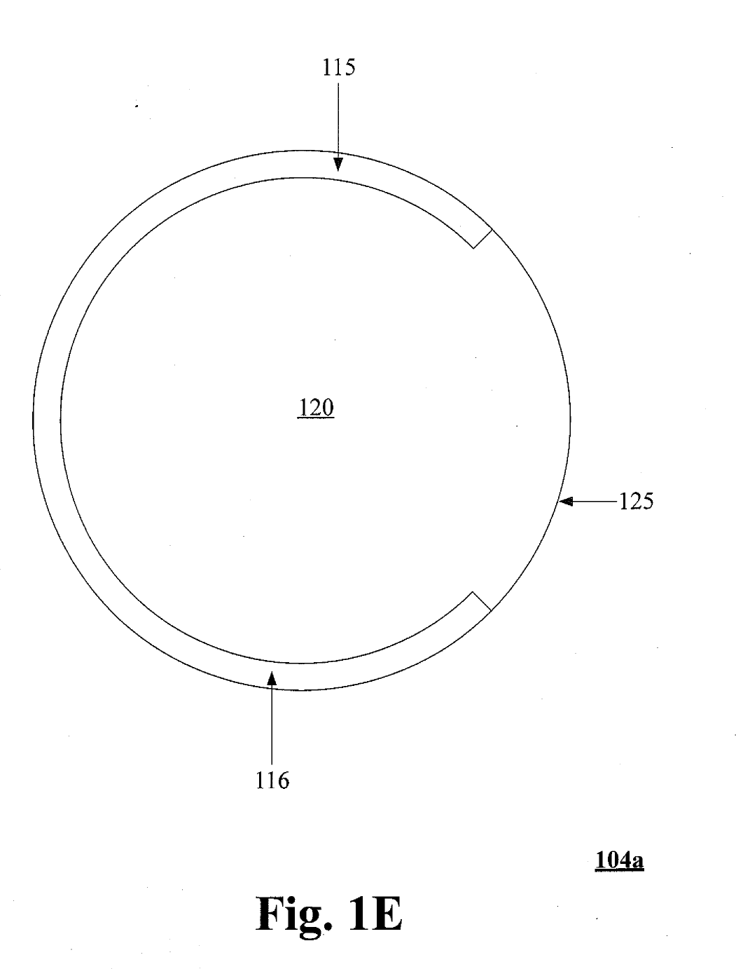

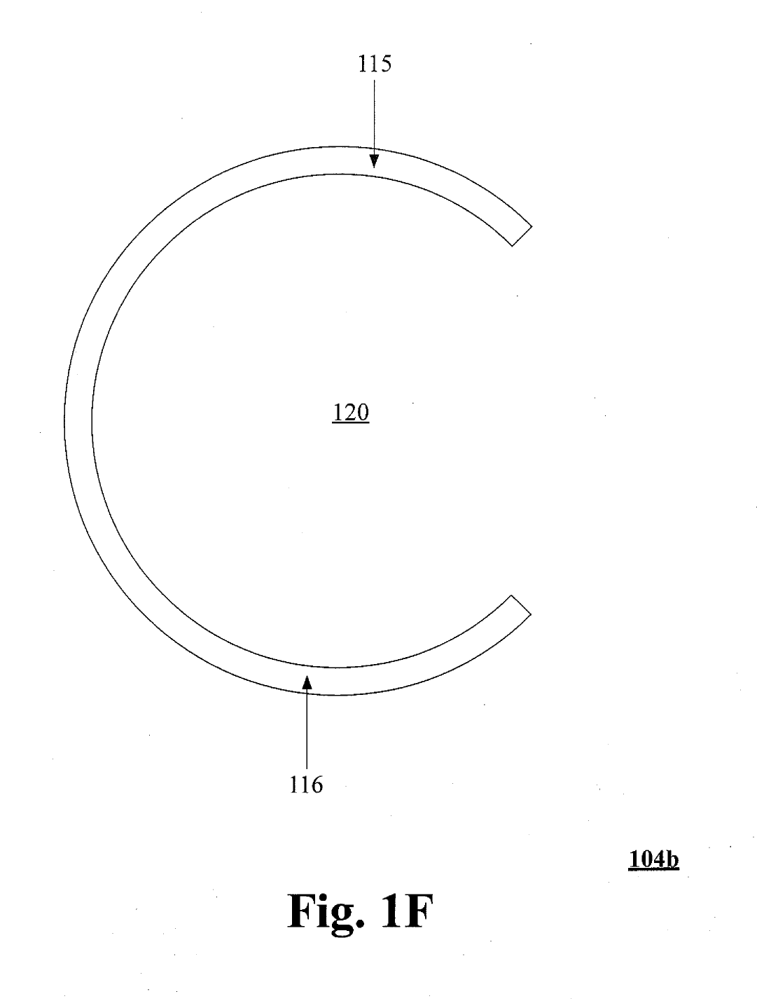

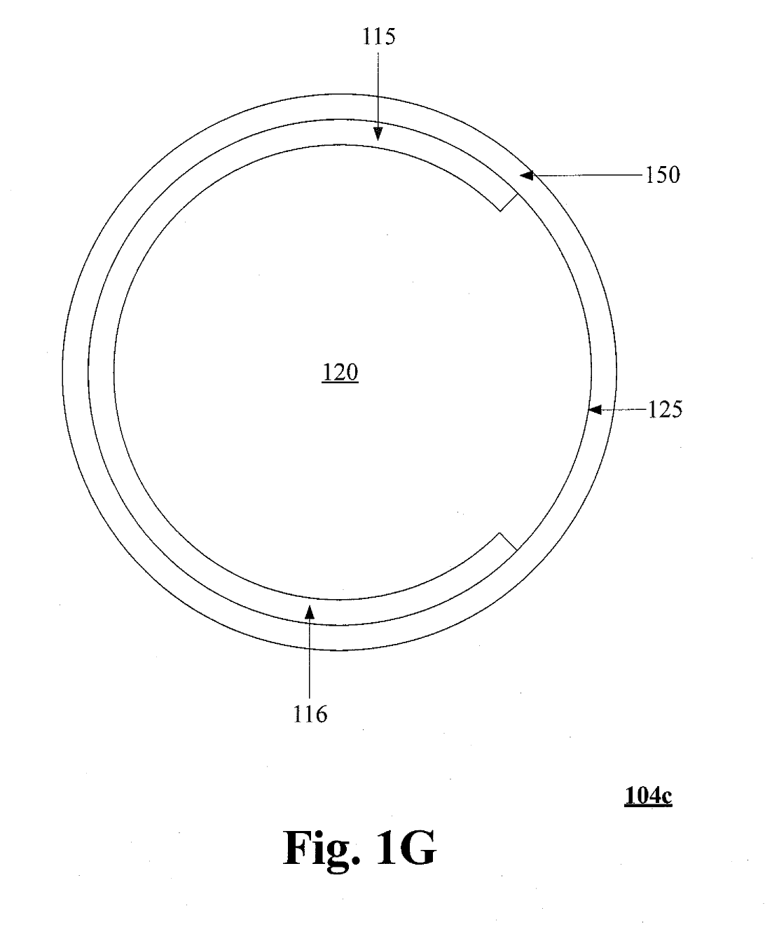

FIG. 1A depicts a side view schematic drawing of an embodiment 100 of a stopper of the invention, said stopper having top 105, sidewalls 115, and 116, and bottom 106, each of which are penetrable by a tip end of a proximal needle (not shown) defining therebetween sequestration chamber 120 at sub-atmospheric pressure.

FIG. 1B depicts a side view schematic drawing of an embodiment 101 of the invention, said stopper having top 105, sidewalls 115 and 116, and bottom 106, defining sequestration chamber 120 therebetween at sub-atmospheric pressure, and showing proximal needle 135 inserted through top 105 and tip end 140 depicted in sequestration chamber 120. The stopper is depicted sealably inserted into a sample container 150, creating a sealed sequestration chamber 155 with sub-atmospheric pressure therein.

FIG. 1C depicts a side view schematic drawing of an embodiment 102 of the invention, with top 105 and bottom 106 of the stopper penetrated by the tip end 140 of proximal needle 135, said tip end 140 shown within space 155 of collection container 150.

FIG. 1D depicts a side view schematic drawing of an embodiment 103 of the invention, with top 105, sidewall 116, and bottom 106 of the stopper penetrated by the tip end 141 of sampling needle 136, said needle being inserted through the stopper without penetrating with sequestration chamber 120 and into space 155 of collection container 150. A sample of relatively uncontaminated fluid can be recovered. Flange 110 is shown and prevents the stopper from being drawn into collection container 150.

FIG. 1E depicts a top view schematic drawing of an embodiment 104a of this invention, depicting sidewalls 115 and 116 and a transparent side window 125, through which an operator or phlebotomist can observe blood or fluid enter sequestration chamber 120.

FIG. 1F depicts a top view schematic drawing of an embodiment 104b of the invention, depicting sidewalls 115 and 116 and a gap, through which an operator or phlebotomist can observe blood or fluid enter sequestration chamber 120.

FIG. 1G depicts a top view schematic drawing of an embodiment 104c of the invention, being inserted into a transparent collection container 150, and depicting sidewalls 115 and 116, and a gap in said stopper being adjacent to container 150 through which an operator or phlebotomist can observe blood or fluid enter sequestration chamber 120.

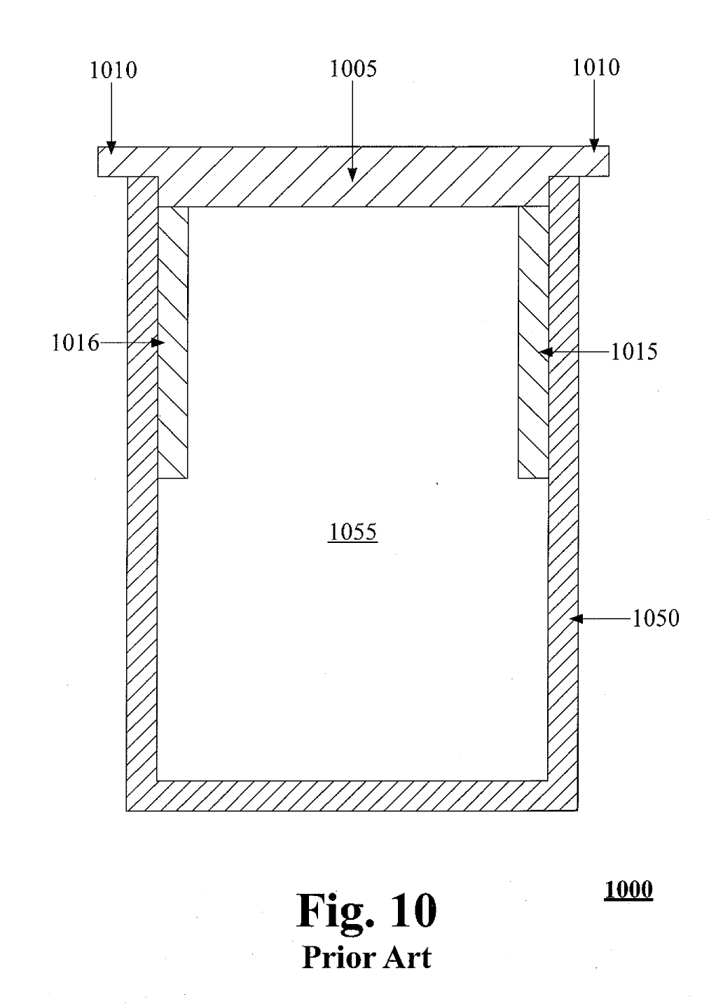

Maintaining a sequestration chamber within a stopper at sub-atmospheric pressure represents a completely new and innovative design for a stopper. Prior art stoppers do not have a sealed sequestration chamber, and thus, when inserted into a vacuum container or other sample collection container, the pressure in the sequestration chamber becomes the same as the pressure in the container or collection device. See Prior Art, FIG. 10. In the prior art, the proximal end of a collection needle would deliver the first amount of fluid drawn directly into the collection container, thereby contaminating the sample.

In contrast, stoppers of the present invention have a bottom portion that, along with the sidewalls and top portion, define a sequestration chamber that can be held at sub-atmospheric pressure, independently of the pressure in the collection container. When in use, the tip end of a proximal needle is inserted through the top portion of the stopper, the first portion of fluid is drawn into the sequestration chamber within the stopper. Subsequently, when the tip end of the proximal needle is inserted through the bottom portion of the stopper, the fluid drawn into the collection container does not include the contaminated portion of the sample, which remains within the sequestration chamber of the stopper. Thus, when analysis of the collected sample is performed, there is a much greater degree of purity of the sample, and results obtained more closely reflect the true values of analytes present in the blood or other bodily fluid.

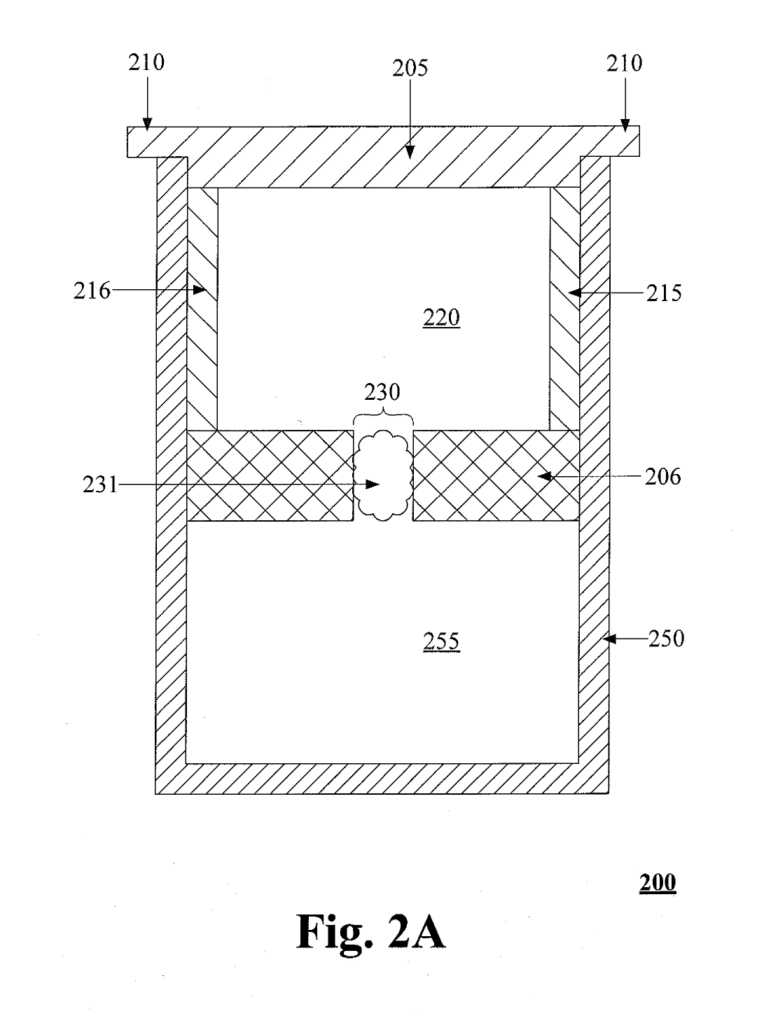

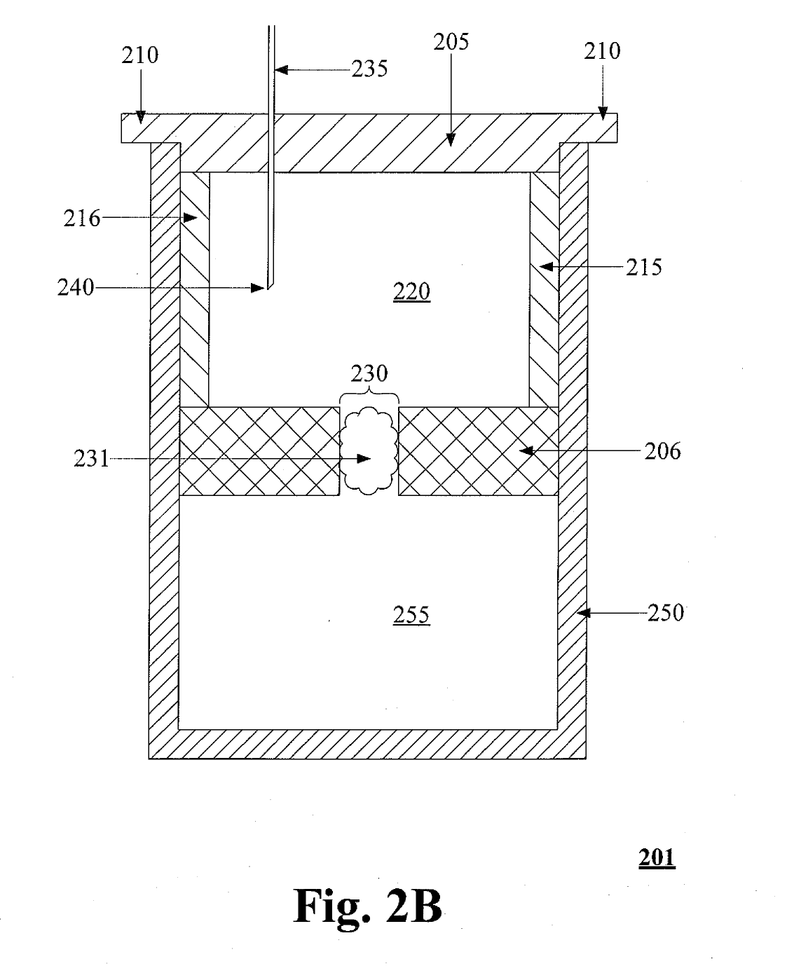

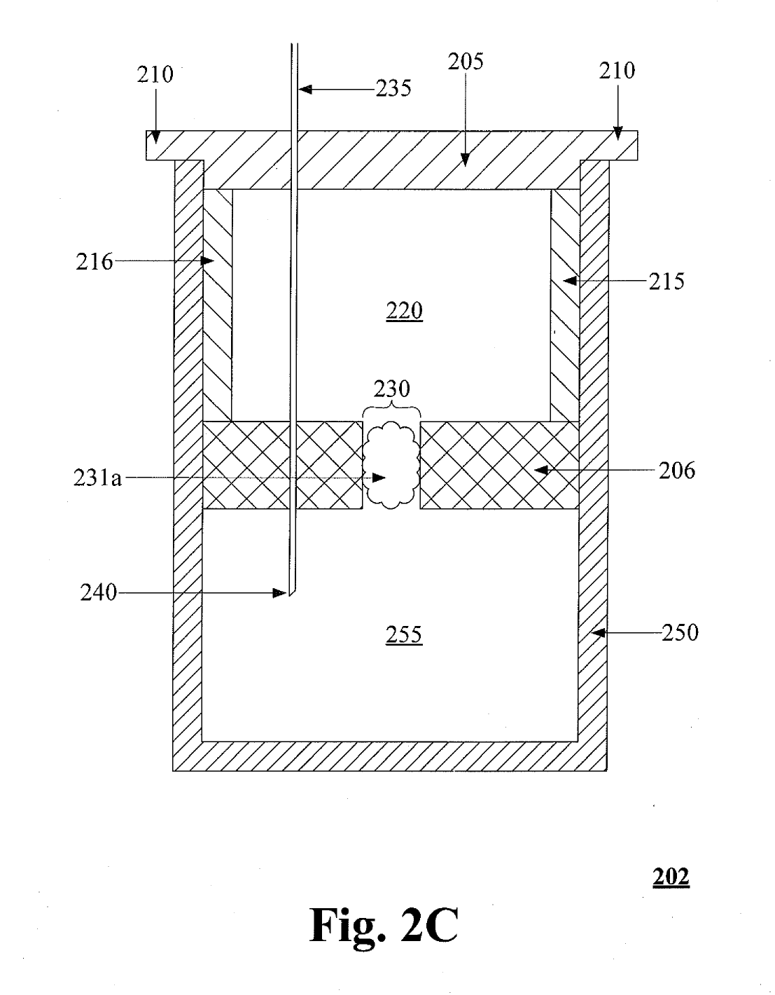

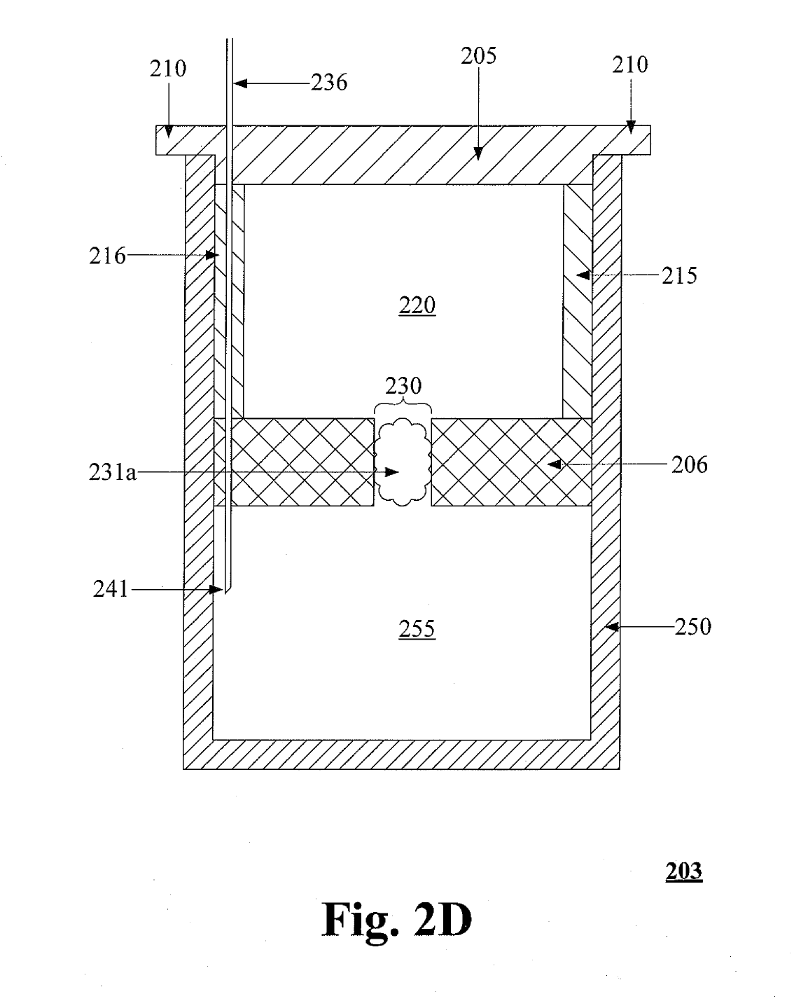

Further aspects include a device according to an additional aspect. As shown in FIG. 2A, wherein sequestration chamber 220 within a stopper is formed between two cylindrical parts 205 and 206 of stopper 200 separated by relatively rigid and resilient parts of the stopper, which further contains an opening 230 of circular, oval, or other form, extending from sequestration chamber 220 to bottom 206, the opening 230 being filled with a suitable absorbent material 231 of suitable porosity or fiber density that when wetted by blood or fluid, becomes 231a, and is relatively impermeable to gas and the fluid wetting or absorbed in it, thus hindering the flow of gas and blood or fluid through it.

Further aspects comprise a device according to a prior aspect, shown in FIG. 2A wherein openings 230 filled with absorbent material can be single, multiple circular, at the periphery of a stopper, or arranged in any suitable number around the periphery of a stopper to guarantee that regardless of the position of the collection container (tube) during blood or fluid drawing, some of the openings 230 will remain dry and therefore gas permeable until sequestration chamber 220 is sufficiently filled with blood or other fluid.

FIG. 2B depicts a side view schematic drawing of an embodiment 201 of the invention as depicted in FIG. 2A with a stopper penetrated by tip end 240 of proximal needle 235, said tip being inserted into the stopper into sequestration chamber 220.

FIG. 2C depicts a side view schematic drawing of an embodiment 202 of FIG. 2B stopper, said absorbent occluding material 231 of FIG. 2B now becoming wetted thereby becoming occlusion material 231a, thereby causing occlusion of opening 230, said closure sealing the stopper sequestration chamber 220 from space 255 in collection container 250, said top 205 penetrated by tip end 240 of proximal needle 235, penetrating bottom 206, with the tip positioned in space 255 of collection container 250, enable the drawing and storing of a test sample of blood or other bodily fluid, with reduced levels of contamination.

FIG. 2D depicts a side view schematic drawing of an embodiment 203 of the invention as shown in FIG. 2C, where said absorptive material 231, now being wetted by the first portion of the sample of fluid, swells and forms occlusion material 231a, thereby occluding opening 230. In this embodiment, a sample collecting needle 236 is shown inserted through top 205, through sidewall 216, and positioned with tip end 241 within space 255 of collection container 250. In this configuration, tip end 241 is in the relatively uncontaminated fluid within the collection container, thereby enabling the drawing and storing elsewhere (not shown) of a test sample of blood or other bodily fluid, with reduced levels of contamination.

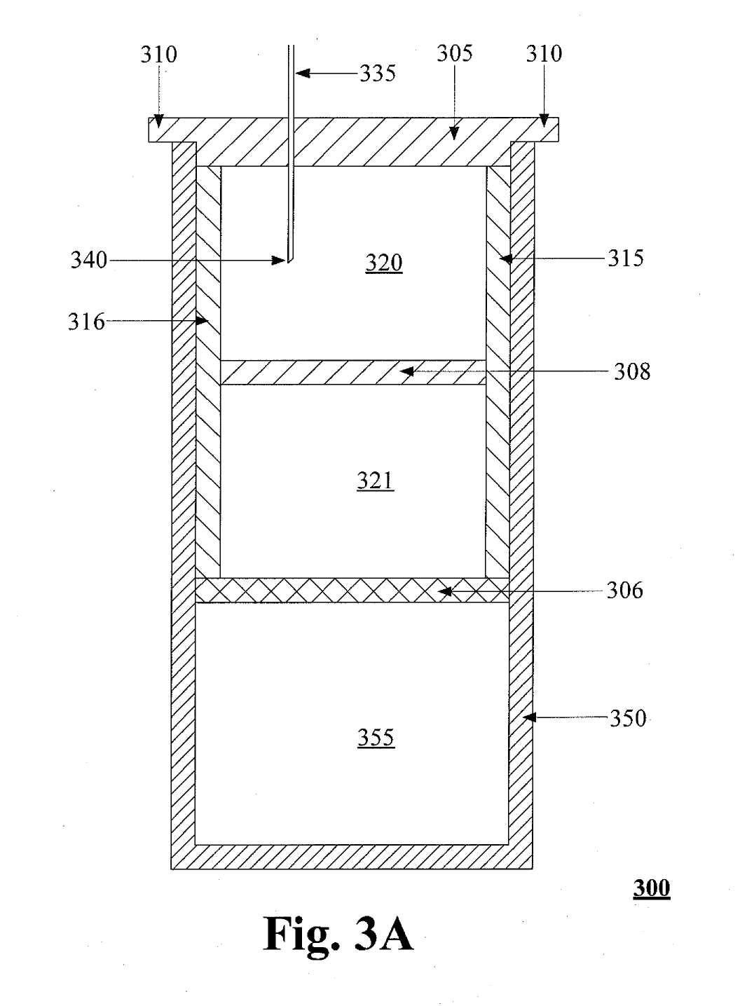

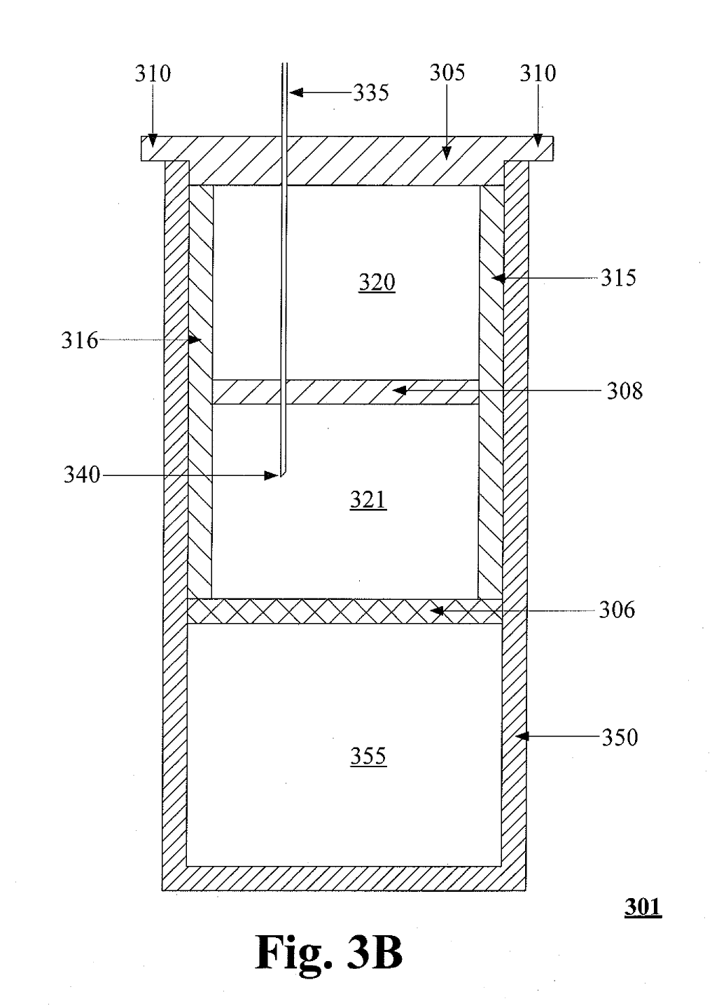

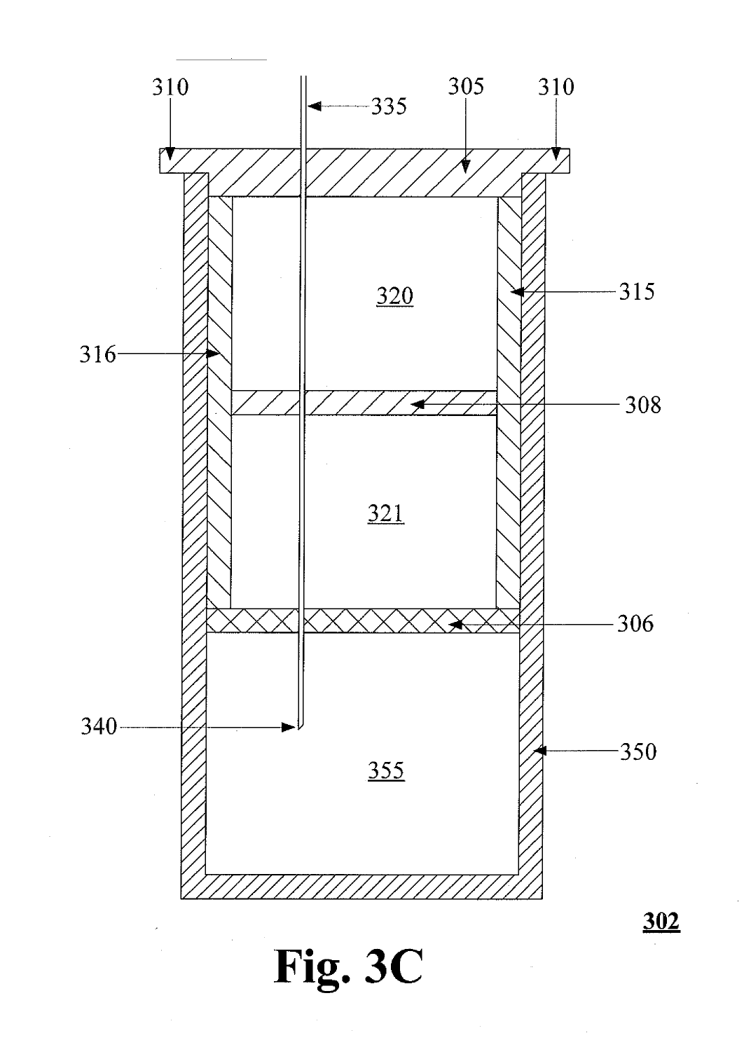

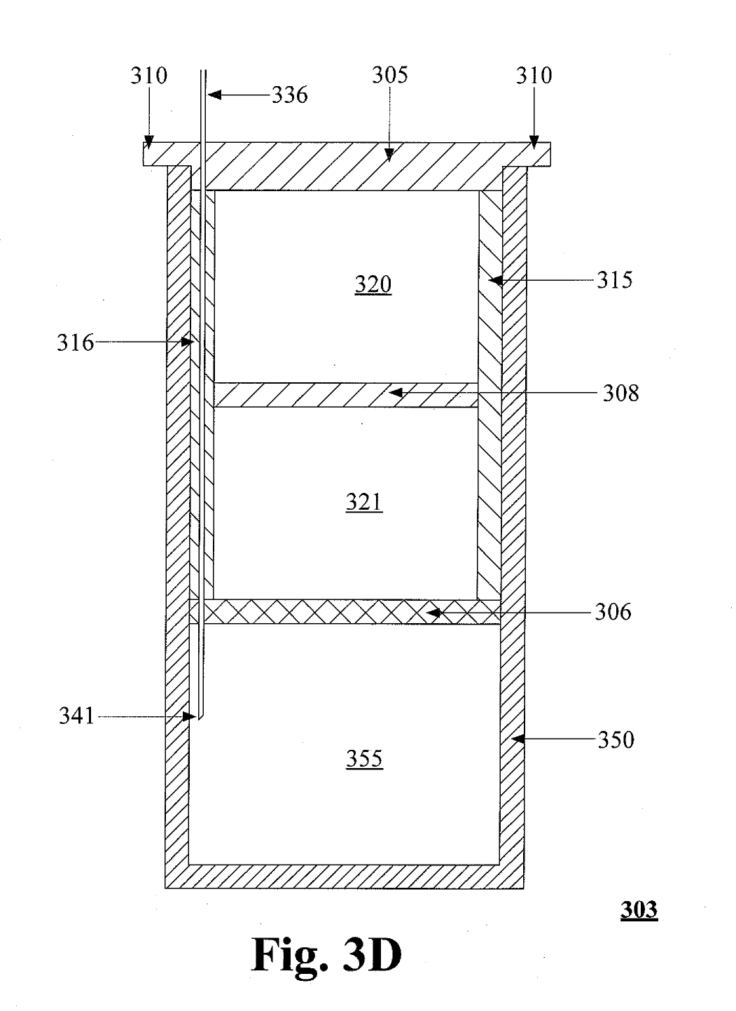

Further aspects include a device as shown in FIG. 3A, having top 305, a plurality of stacked sequestration chambers 320 and 321, each containing a side wall 315 and a side wall 316, with sequestration chamber 320 containing a bottom 308 that is needle penetrable, said bottom layer 308 being the top layer for the next sequestration chamber 321, said sequestration chamber 321 having a bottom 306, also penetrable by a needle. It can be appreciated that there can be other embodiments having more than two such sequestration chambers. Also, the sequestration chambers may have different configurations to ease operators' need for accuracy in sequestering a first, second, or additional samples.

In FIG. 3A, proximal needle 335 is shown penetrated through top 305, and tip end 340 positioned within sequestration chamber 320. A second sequestration chamber 321 is shown, having a bottom 306 and space 355 within collection container 350.

FIG. 3C depicts a side view schematic drawing of an embodiment 301 of this invention as shown in FIGS. 3A and 3B, but with proximal needle 335 inserted through bottom 308 and into space 355 of collection container 350.

FIG. 3D depicts a schematic drawing of an embodiment 303 of this invention as shown in FIGS. 3A, 3B, and 3C, with a sample collection needle 335 inserted through top 305, through sidewall 316, with tip end 341 located within space 355 of collection container 350. With this configuration, a sample of fluid (not shown) within space 355 can be obtained with contaminants being sequestered in sequestration chambers 320 and 321.

Use of stacked sequestration chambers as depicted in FIGS. 3A, 3B, 3C, and 3D can decrease contamination of a sample, and thereby deliver a sample much greater degree of purity of the sample, and results obtained more closely reflect the true values of analytes present in the blood or other bodily fluid.

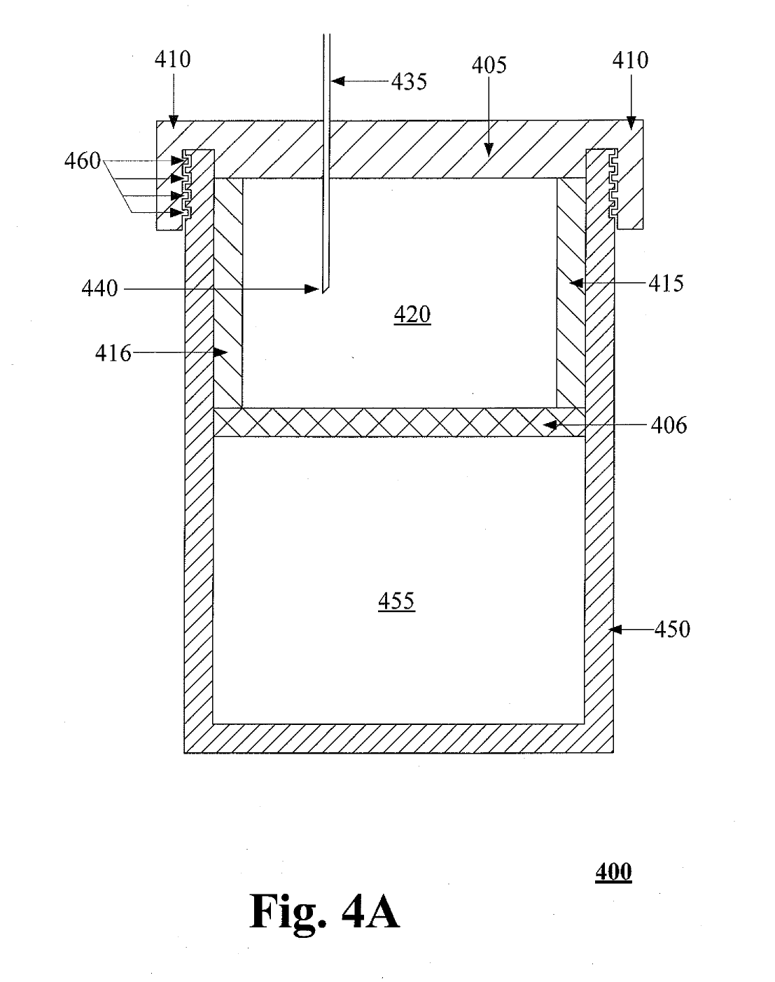

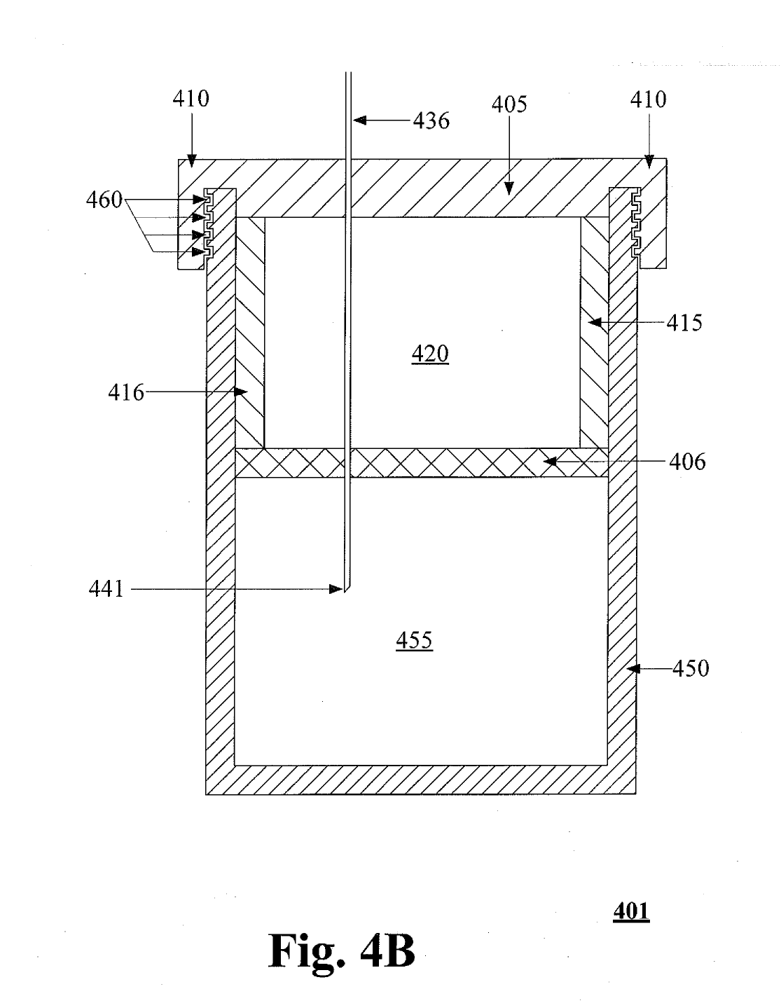

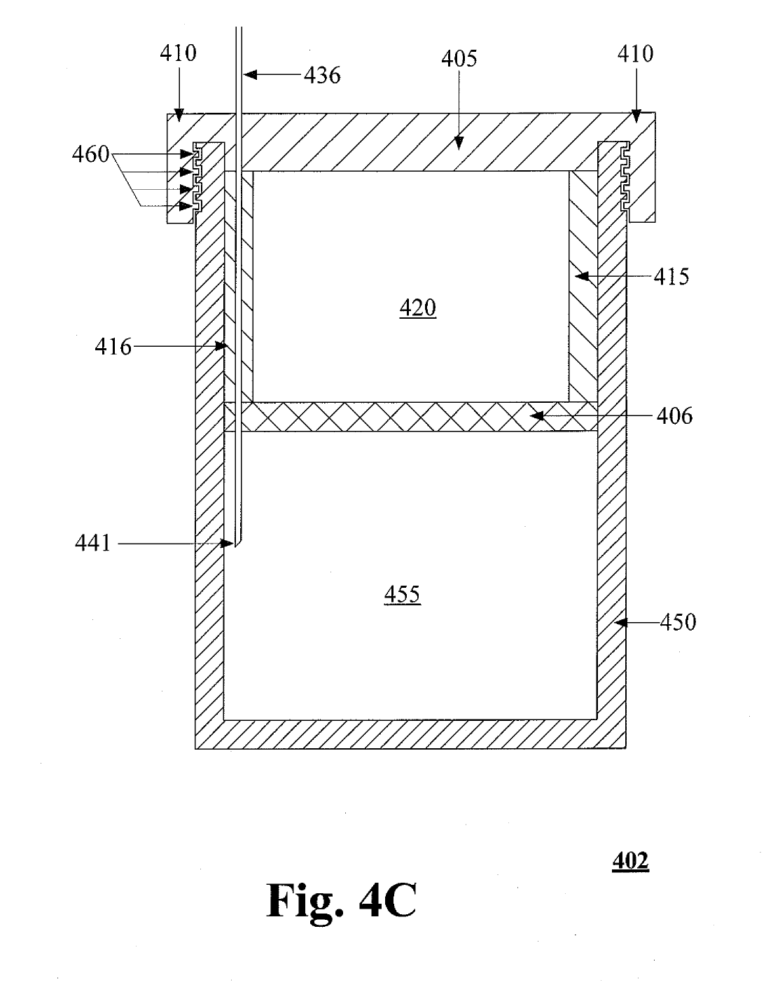

Further aspects include a device according to a prior aspect, shown in FIG. 4A, depicts a side view schematic drawing of an embodiment 400 of a screw cap stopper of the invention, with threads and groves 460, said screw cap threadably engaged with top end of container 450 and creating a liquid and gas sealed space 455 within collection container 450, Screw cap stopper contains sequestration chamber 420 at sub-atmospheric pressure. Sequestration chamber 420 contains a side walls 415 and 416 and bottom 406, wherein the sub-atmospheric pressure can draw a first portion of fluid collected into sequestration chamber 420.

FIG. 4B depicts a side view schematic drawing of an embodiment 401 of the invention where proximal needle 436 is shown penetrating top 405, and bottom 406, and tip end 441 being located within space 455 of collection container 450.

FIG. 4C depicts a side view schematic drawing of an embodiment 402 of the invention as shown in FIGS. 4A, and 4B where the sample collection needle 436 is shown after passing through top 405, sidewall 416 and bottom 406, with tip end 441 of proximal needle 436 being located within space 455 of collection container 450. When so positioned, sample collection needle. This configuration enables the drawing and storing of a test sample of blood or other bodily fluid elsewhere, with reduced levels of contamination.

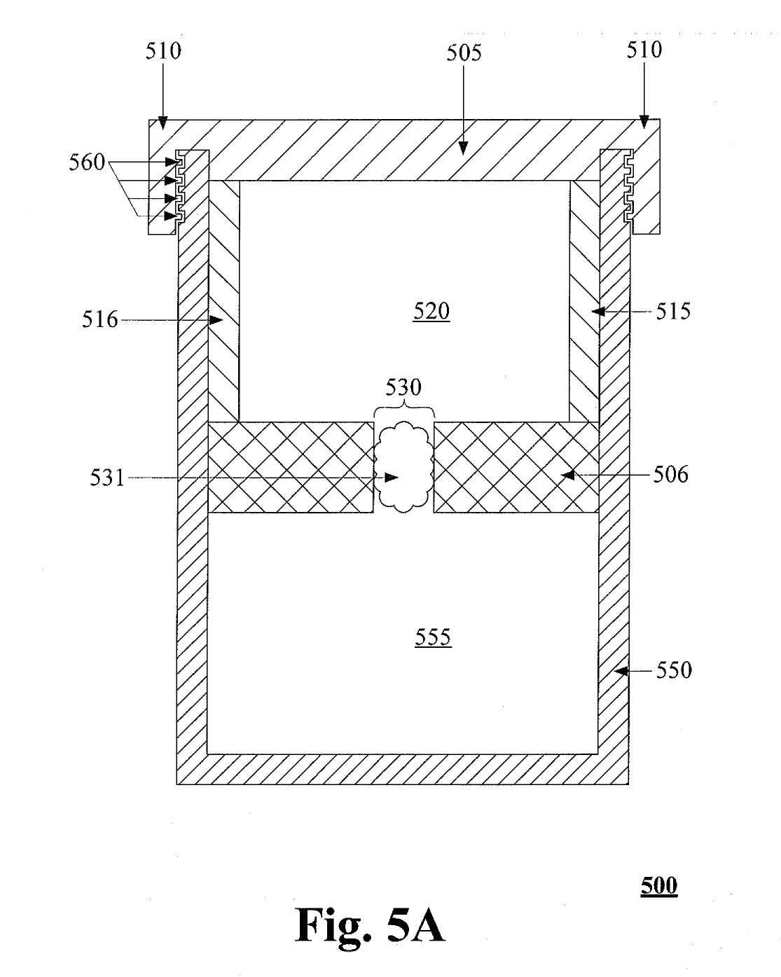

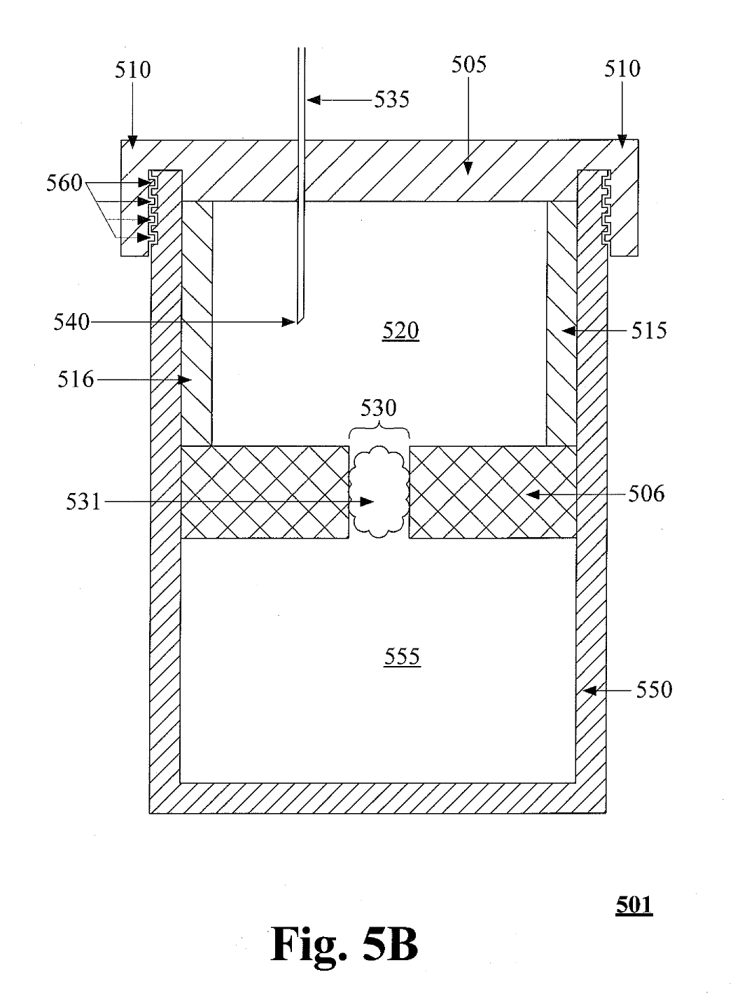

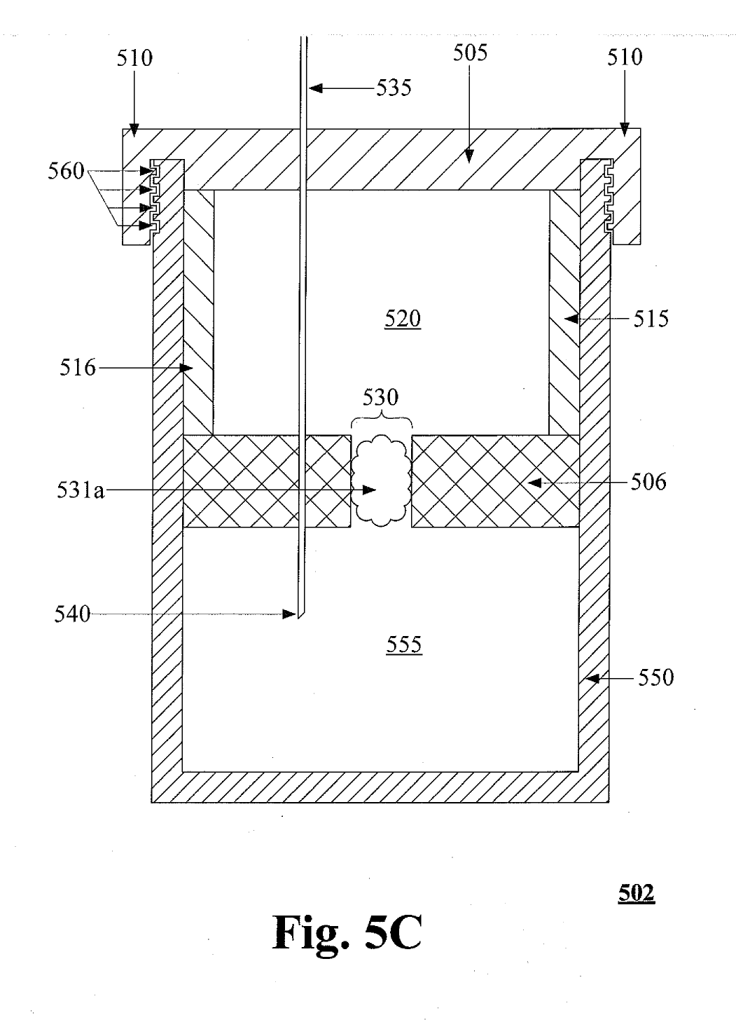

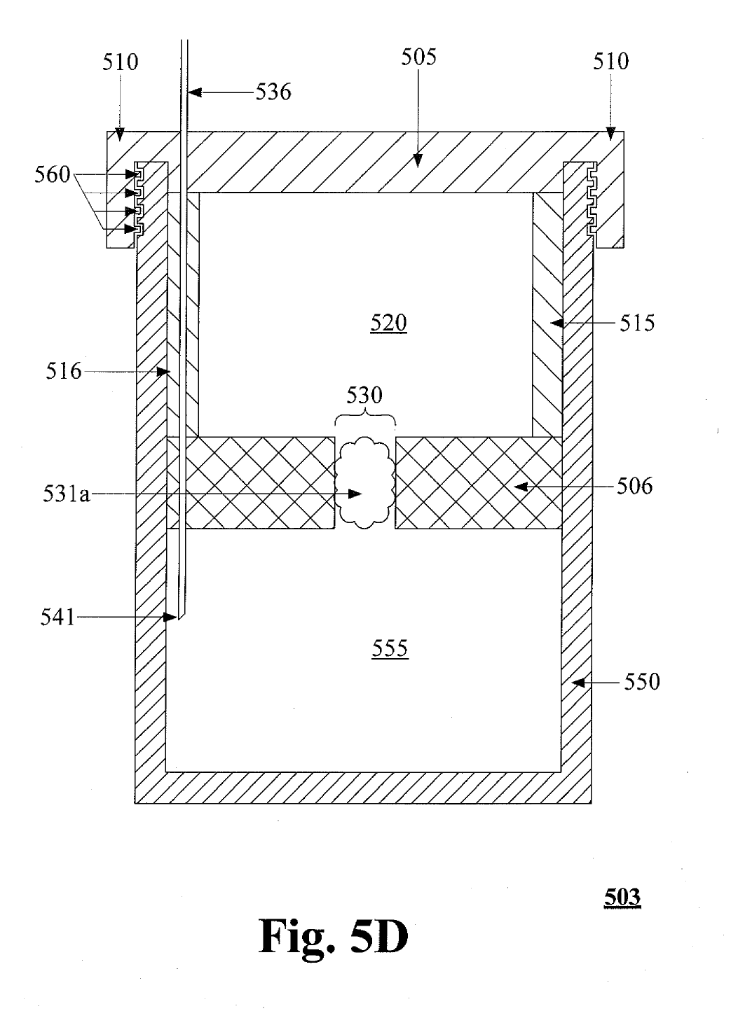

Further aspects include a device of this invention as shown in FIG. 5A. FIG. 5A depicts a side view schematic drawing of an embodiment 500 of a screw cap stopper of the invention, said screw cap stopper attached to a collection container 550 creating a sealed space 555 within collection container 550. The screw top stopper contains a sequestration chamber 520, which is connected to space 555 of collection container 550 by opening 530, with absorbent, occlusion-creating material 531 therein.

FIG. 5B depicts a side view schematic drawing of an embodiment 501 of the invention as shown in FIG. 5A, but wherein proximal needle 535 is shown penetrated through top 505, and tip end 540 of proximal needle 535 shown within sequestration chamber 520. Opening 530 is shown, with absorbent occlusion material 531 being not wetted, and therefore not occluding opening 530.

FIG. 5C depicts a side view schematic drawing of an embodiment 502 of the invention as shown in FIGS. 5A and 5B, with proximal needle 535 shown penetrated through top 505, and bottom 506. As tip end 540 passed into sequestration chamber 520, the first portion of a sample was deposited therein, and fluid in the sample wetting absorbent occluding material 531a which swells, thereby occluding opening 530, sealing sequestration chamber 520 from space 555. With tip end 540 within space 555, the tip is in position to deposit a relatively uncontaminated portion of the fluid into space 555.

FIG. 5D depicts a side view schematic drawing of an embodiment 503 of this invention, with sample needle 535 shown penetrated through top 505, sidewall 516, through bottom 506. Tip end 541 is shown within space 555 of collection container 550. As the first portion of the fluid was sequestered in sequestration chamber 520, the fluid wetted absorbent material 531 causing it to swell and occlude opening 530, effectively sealing sequestration chamber 520 from space 555. Tip end 541 of sampling needle 531 is shown in the fluid (not shown) within space 555. This configuration enables the drawing and storing of a test sample of blood or other bodily fluid elsewhere, with reduced levels of contamination.

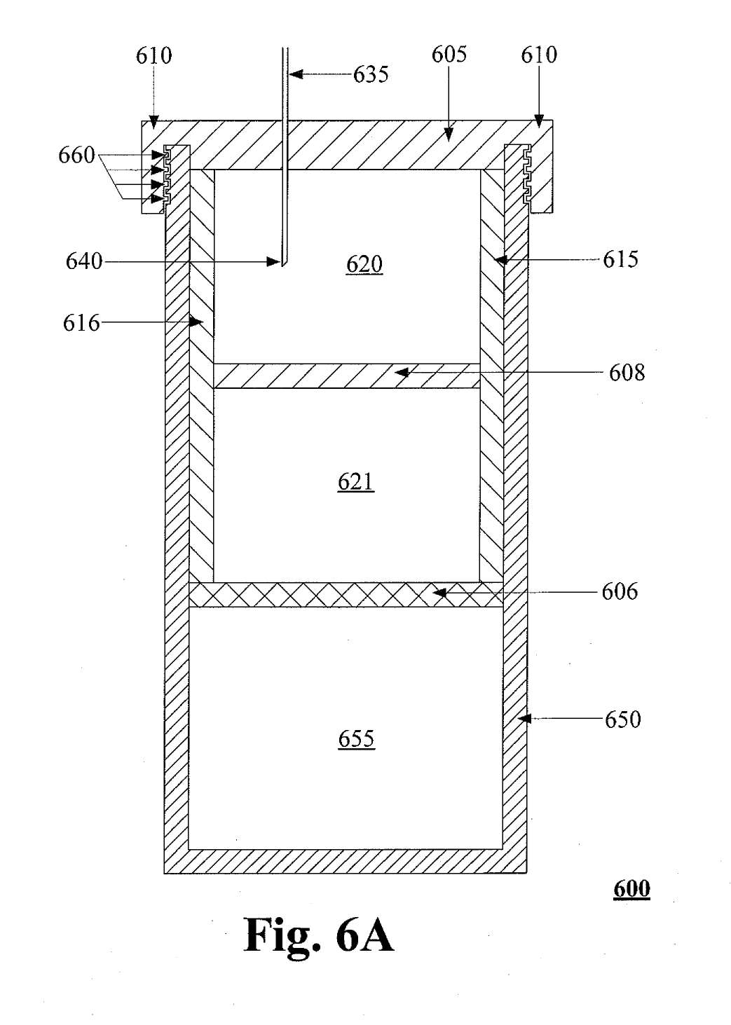

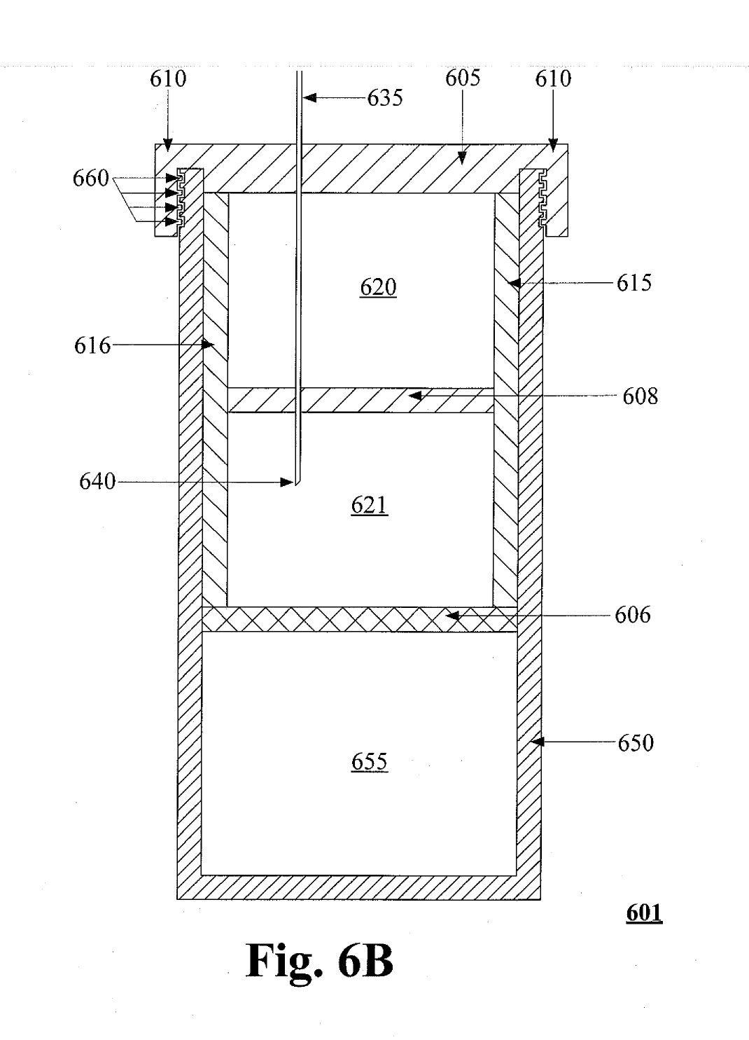

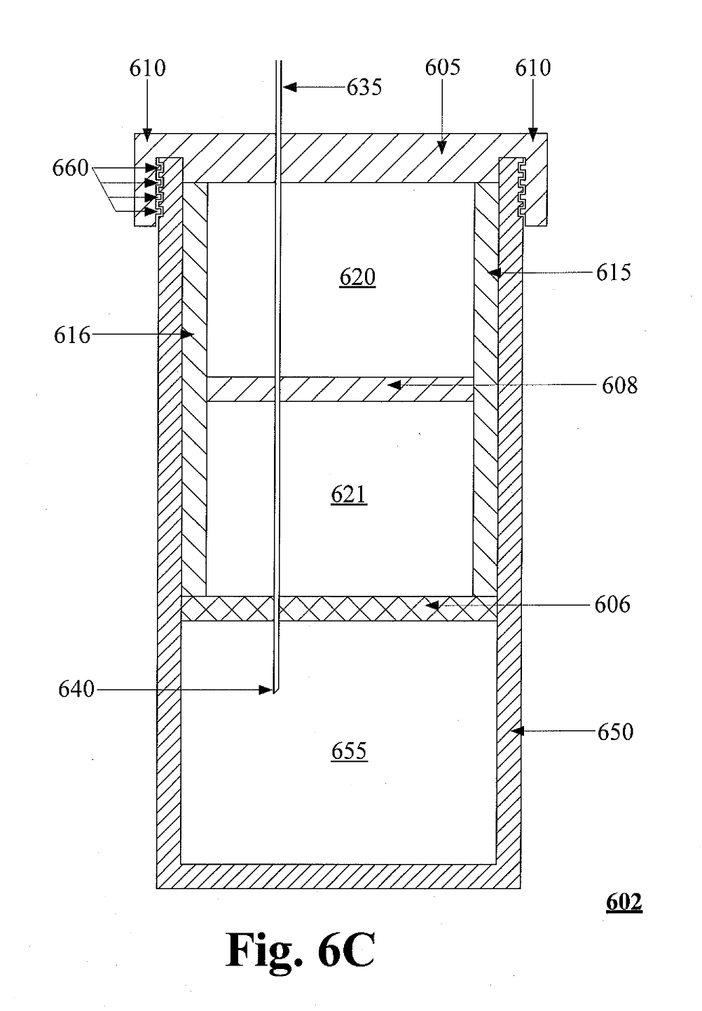

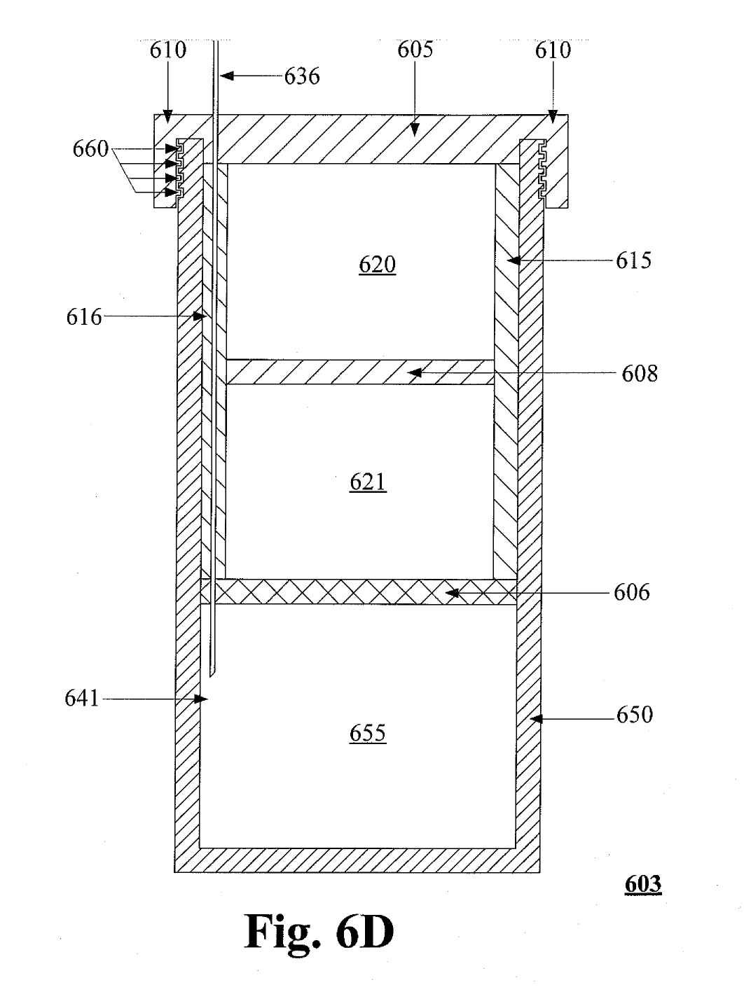

FIG. 6A depicts a side view schematic drawing of an embodiment 600 of this invention, similar to that shown herein, having a screw cap stopper of the invention as described herein, the stopper containing two stacked, sequestration chambers 620 and 621 each at sub-atmospheric pressure.

Proximal needle 635 is shown penetrated through top 605, with tip end 640 of proximal needle 635, shown within sequestration chamber 620. Also shown is a second, stacked sequestration chamber 621 having bottom 606 and space 655 within collection container 650.

FIG. 6B depicts a side view schematic drawing of an embodiment 601 of this invention as shown in FIG. 6A, but where proximal needle 635 show penetrated through top 605, and bottom 608 of the upper sequestration chamber 620. Tip end 640 is shown within sequestration chamber 621.

FIG. 6C depicts a side view schematic drawing of an embodiment 602 of this invention, where proximal needle 635 is shown penetrated through top 605, bottom 608 of sequestration chamber 620, and through bottom 606 of sequestration chamber 621. Tip end 640 of proximal needle 635 is shown within space 655 of collection container 650. In this position, the first portion of the sample has been sequestered in sequestration chamber 620, a second portion of the sample has been sequestered in sequestration chamber 621, and the remainder of the sample, being relatively uncontaminated is deposited in space 655 of collection container 650.

FIG. 6D depicts a side view schematic drawing of an embodiment 603 of this invention as shown in FIG. 6C, but having a sampling needle 636 shown penetrated through top 605, sidewall 616, and bottom 606 of the lower sequestration chamber 621. Tip end 641 of sampling needle 636 is shown in space 655, and within the sample of fluid (not shown). In this configuration, sampling needle 636 can be used to withdraw and store elsewhere a relatively uncontaminated portion of fluid from space 655 of collection container 650.

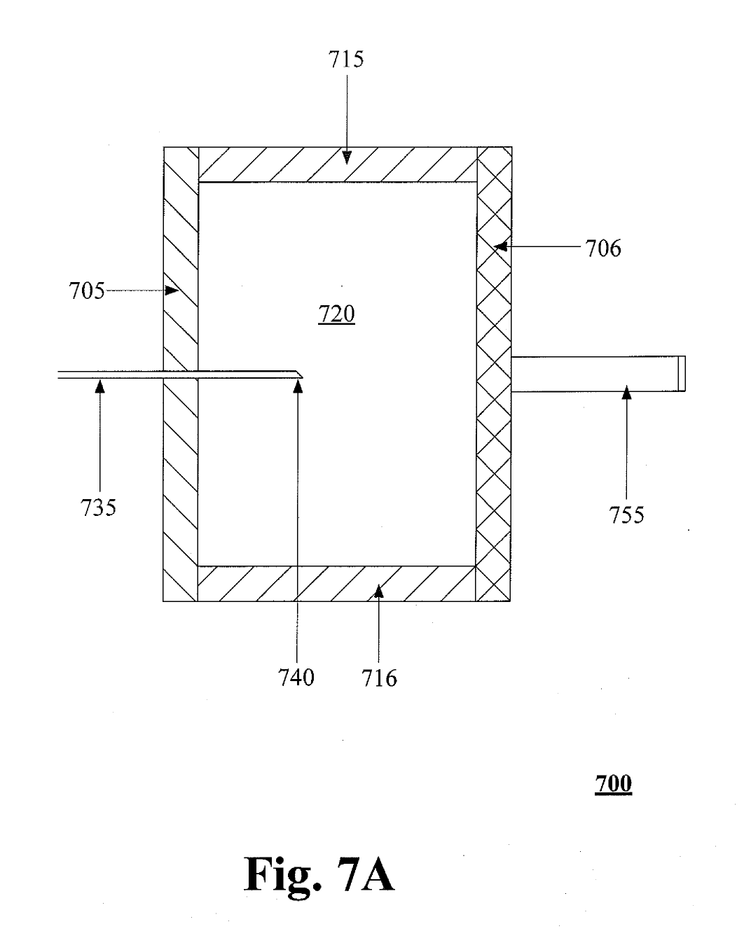

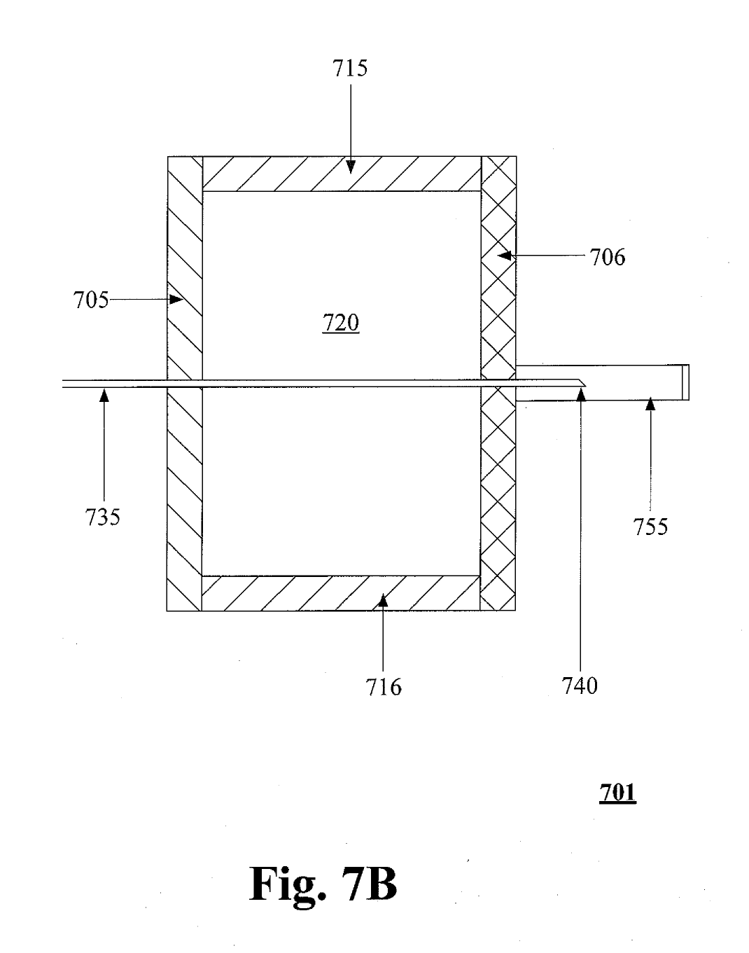

Further aspects include an “in-line” embodiment of a device of this invention shown in FIG. 7A. FIG. 7A depicts a side view schematic drawing of an embodiment 700 of an in-line embodiment of the invention, containing left end 705, right end 706, top end 715, and bottom side 716 defining sequestration chamber 720 at sub-atmospheric pressure, penetrated by proximal needle 735, with tip end 740 of proximal needle 735 shown within sequestration chamber 720. Outflow tube 755 is shown attached to the outer portion of right sidewall 706. Left sidewall 705 and right sidewall 706 are made of a resilient material penetrable by a needle. Top 705, sidewalls 705 and 706, and bottom 716 are made of a material that is sufficiently rigid to withstand sub-atmospheric pressures of from about 1% to about 90% of the ambient atmospheric pressure. In this configuration, a first portion of a sample can be sequestered within sequestration chamber 720.

FIG. 7B depicts a side view schematic drawing of in-line embodiment 701 of this invention, but where tip end 740 of proximal needle 735 is shown penetrating through right sidewall 706 and into collection tube 755.

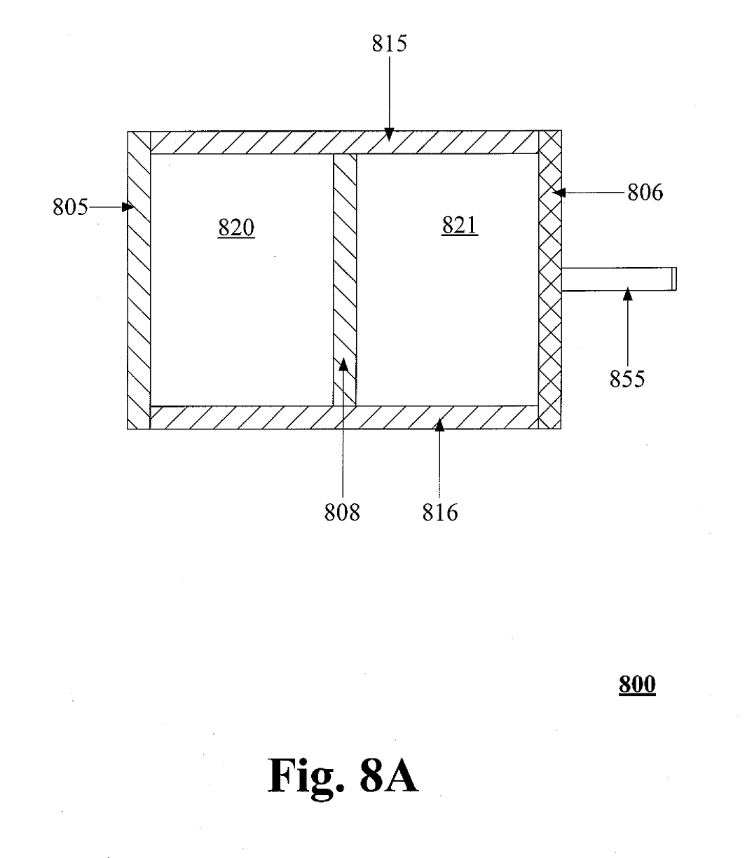

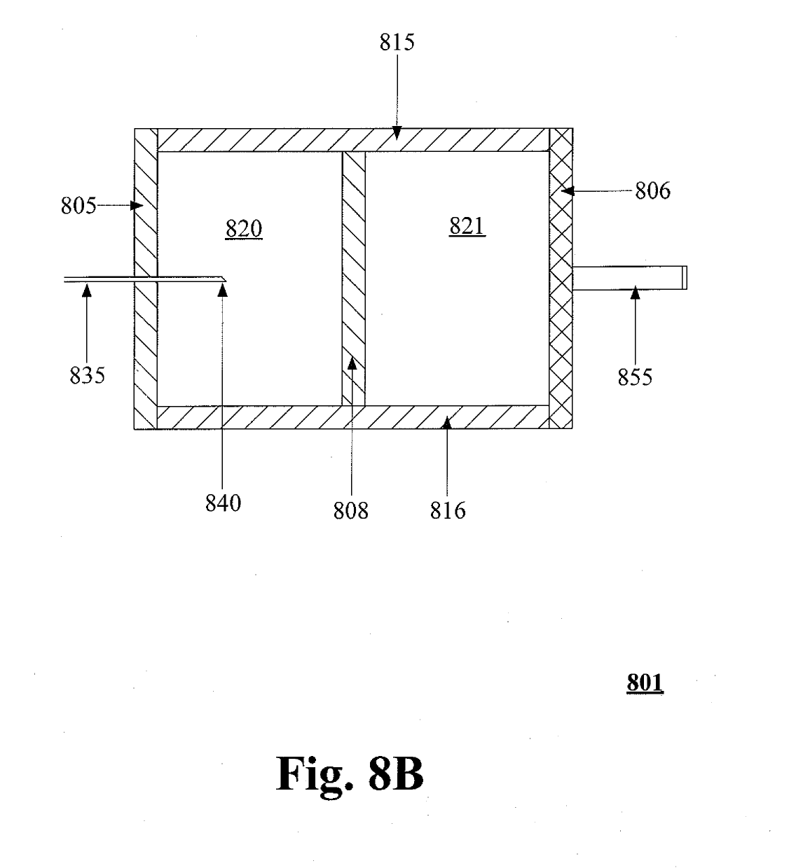

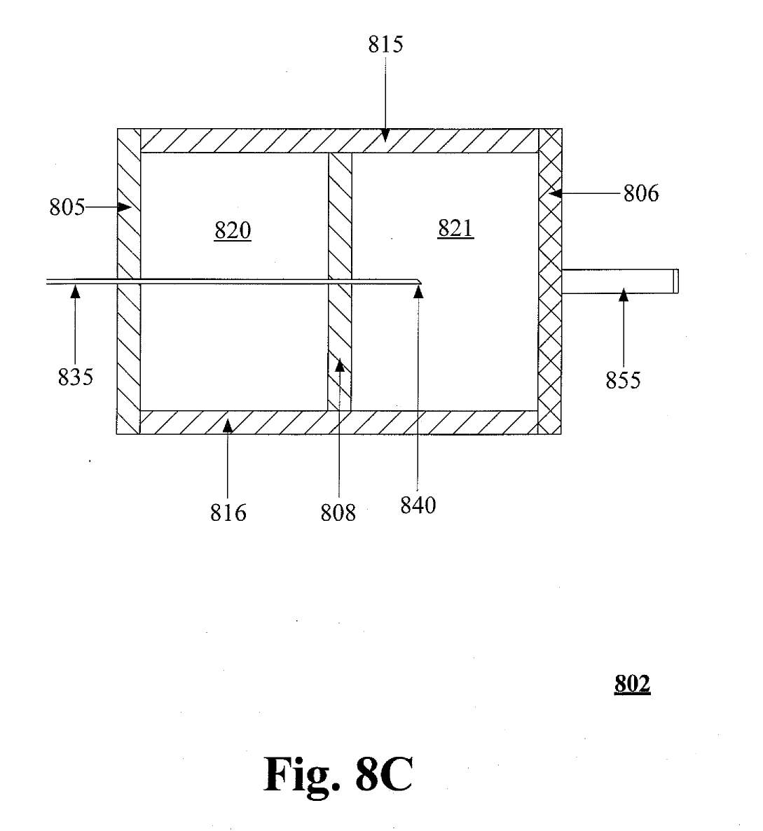

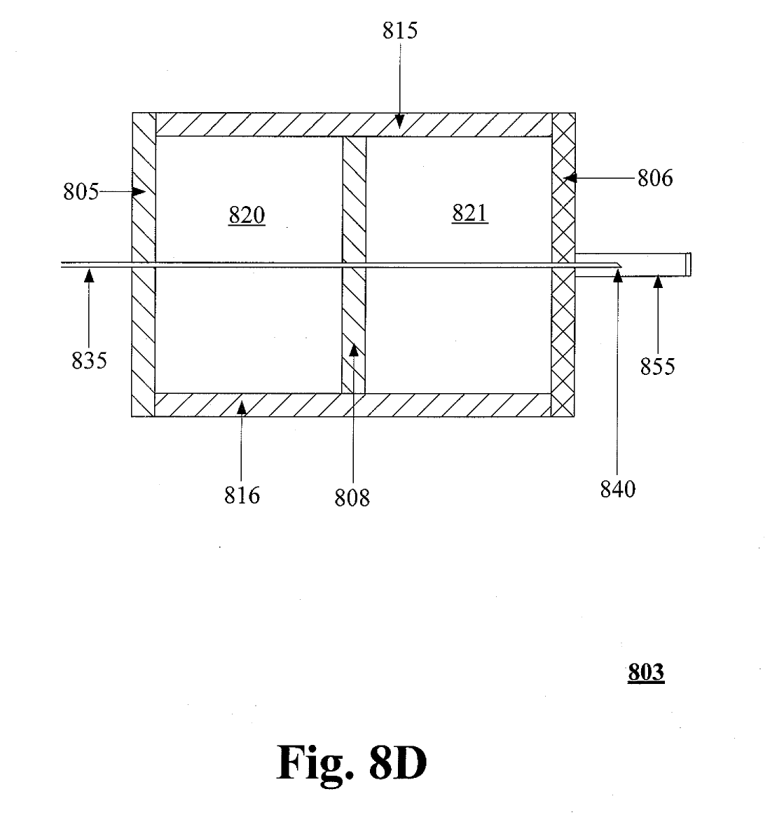

FIG. 8A depicts a side view schematic drawing of an in-line embodiment 800 of the invention, similar to that shown in FIGS. 7A and 7B, but comprising two stacked sequestration chambers 820 and 821, each at sub-atmospheric pressure. Left sidewall 805, middle wall 808, right sidewall 806, top 815, and bottom 816 are shown with sequestration chambers 820 and 821 therebetween. Outflow tube 755 is shown affixed to the outer face of right sidewall 806, said embodiment connected to an out flow tube 855.

FIG. 8B depicts a side view schematic drawing of an embodiment 801 of the invention as shown in FIG. 8A, where sequestration chambers 820 is penetrated by tip end 840 of proximal needle 835. In this configuration, a first portion of a sample is drawn into sequestration chamber 820 where it is sequestered.

FIG. 8C depicts a side view schematic drawing of an in-line embodiment 802 similar to that shown in FIG. 8B, where sequestration chambers 820 and 821 are penetrated by proximal needle 835. Tip end 840 is shown within sequestration chamber 821, where a second portion of the sample is drawn into sequestration chamber 821, where it is sequestered.

FIG. 8D depicts a side view schematic drawing of an in-line embodiment 803 similar to that shown in FIG. 8C, where in-line sequestration chambers 820 and 821 are penetrated by proximal needle 835, and tip 840 of proximal needle 835 shown penetrated through right sidewall 806 and into out flow tube 855.

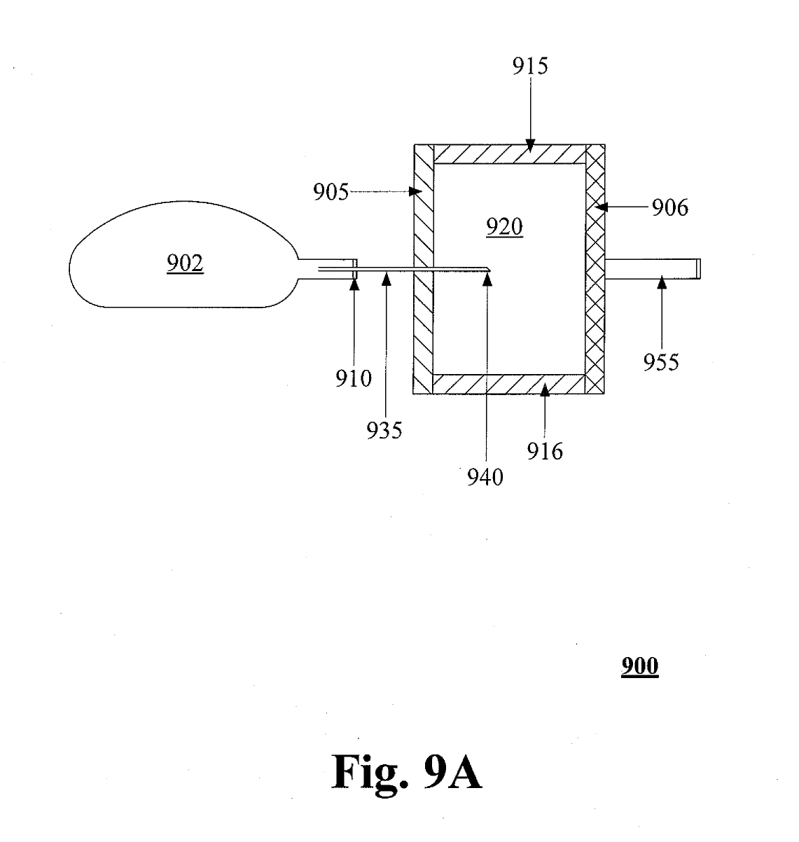

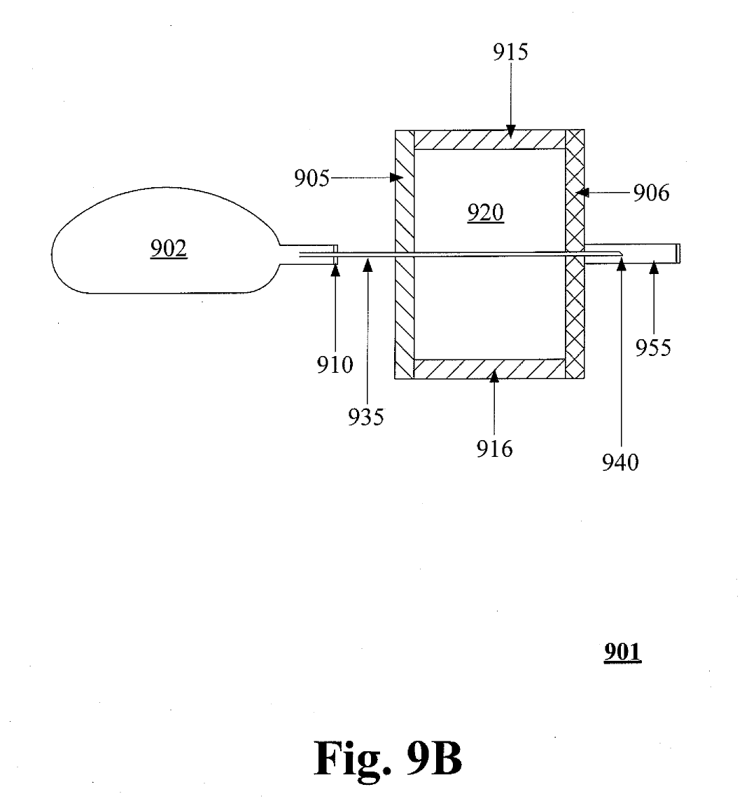

Exemplary uses of devices as shown in FIGS. 8A-8D are shown in FIG. 9A. FIG. 9A depicts embodiment 900 of this invention, in which an in-line device as described herein is shown adjacent to a culture bag 902. Culture bag 902 may contain cells, tissues, growth medium (fluid), and/or other components used to grow and maintain the living tissue or cells within the culture bag 902. Periodically, it is desirable to sample the medium (fluid) from the bag to test for viability of the cells or tissues, or to measure production of products produced by the cells. However, it is possible that the results of such tests may be compromised by contamination present on the outside of the bag 902, especially where a proximal needle is to be inserted into the bag.

To address this need, this invention contemplates use of “in-line” embodiments to sequester a first portion of a culture sample of fluid within a sequestration chamber. FIG. 9A shows culture bag 902, having port 910 that is penetrable by distal end (the left portion) of proximal needle 935 penetrated through port 910 and in contact with culture fluid within bag 902. Tip end 940 of proximal needle 935 is shown within sequestration chamber 920, where a first portion of the sample is drawn into sequestration chamber 920 where it remains sequestered.

FIG. 913 depicts a schematic drawing of an in-line embodiment 901 of this invention as shown in FIG. 9A, but where right sidewall 906 is penetrated by tip end 940 of proximal needle 935, which is shown in the out flow tube 955. A sample of culture fluid is thereby obtained in outflow tube 955 that is relatively uncontaminated.

FIG. 10 depicts a side view schematic drawing of conventional stopper 1000 of the prior art, having a penetrable top 1105 with flange 1010, sidewalls 1015 and 1016, each made of a resilient material, defining a space between the sidewalls, but without a bottom portion. The stopper is shown sealably inserted within collection container 1050 having sub-atmospheric pressure therein.

In use, the conventional stopper has sub-atmospheric pressure throughout the space 1055. When tip end of a proximal needle (not shown) is inserted through top 1005, the entire sample is drawn into space 1055. Thus, both the first part of the sample, with contaminants, is mixed in space 1055 along with the remainder of the sample.

### Stopper Structure

Stopper 100 shown in FIG. 1A is constructed of resilient material, such as rubber or plastic capable of being punctured by a proximal end of a needle used to draw blood and other bodily fluids as test samples. Stopper 100 contains at least one sequestration chamber 120 capable of holding from 1 cubic millimeter to 100,000 cubic millimeters of blood or other fluid.

Sequestration chamber 120 can be formed between top portion 105, bottom portion 106 and sidewalls 115 and 116. Window 125 of rectangular, circular, oval or any other shape, can be formed through a sidewall, through which an operator or phlebotomist can observe blood or fluid enter sequestration chamber 120. Window 125 is sealingly covered by a transparent wall of collection vessel 150. Volumes, shapes, numbers, and locations of sequestration chambers can easily be determined and adapted to specific needs by anyone skilled in the art.

In certain embodiments of the invention, shown in FIG. 2A, stopper 200 has one or more openings 230 of circular, oval, or other form, extending through bottom 206. Openings 230 are filled with material 231 of such absorbency and effective pore size ranging from 0.02 μm to 1 mm. that when wetted with blood or fluid, the material becomes relatively impermeable to gas and fluid wetting or absorbed in it, thus hindering flow of blood or fluid through openings 230 in stopper 200. The type of material including packed or woven fibers of cotton, cellulose, cellulose fibers and polyamines, cellulose fibers and cationic starch, rayon, cotton, silk, nylon, microporous polyvinylidene difluoride (PVDF), microporous mixed cellulose esters (MCE), and poly tetra fluoro ethylene (PTFE), mixed cellulose esters (MCE), acrylonitrile butadiene styrene (ABS), butadiene styrene rubber (BS), cyclohexanedimethanol (CHDM), cellulose nitrate-cellulose acetate (CN-CA), ethylene propylene terpolymer rubber (EPDM), ethylene vinyl acetate (EVA), high density polyethylene (HDPE), high density polypropylene (HDPP), high impact polystyrene (HIPS), low density polyethylene (LDPE), methyl methacrylate ABS (MABS), nitrite butadiene rubber (NBR), neoprene (NPRN), polyamide (PA), polybutylene terephthalate (PBT), polycarbonate (PC), polyethylene (PE), polyethersulfone (PES), polyethylene terephthalate (PET), PET modified with glycolor (PETG), polyimide (PI), butyl rubber (PIB), polyoxymethylene (POM), polypropylene (PP), polystyrene (PS), polysulfone (PSU), polytetrafluoroethylene (PTFE), polyurethane (PU), polyvinyl acetate (PVA), polyvinyl chloride (PVC), styrene butadiene (SB), styrene butadiene rubber (SBR), stainless steel (SS), thermoplastic elastomer (TPE), thermoplastic rubber (TPR) with an effective pore size ranging from 0.02 μm to 1 mm, mixtures of materials with a pore size ranging from 0.02 μm to 1 mm of uniform or non-uniform hydrophobicity for fitting the closures between sequestration chambers and specimen receptacle can be easily determined and adapted to specific needs by a person of ordinary skill in the art.

Openings 230 extending from sequestration chambers 220 through bottom 206 are filled with absorbent material, and can be single, multiple circular, at the periphery of the stopper or arranged in any suitable number around the periphery of the stopper, such as to guarantee that regardless of the position of sample collection container tube during blood or fluid drawing, one or more openings 230 will remain dry and therefore permeable to gas until sequestration chamber 220 is filled with blood or other fluid.

In certain other embodiments of the invention as shown in FIG. 3A, a stopper has a suitable solid area of continuity between bottom 306 and top 305, through which a sample collection needle 336 can transverse the stopper without touching or going through a sequestration chamber 320. Safety of drawing needle's conduit through the stopper is ensured by use of needle-puncturable material in formation of any access path for a needle through the stopper.

A multiplicity of sequestration chambers 320 can be stacked within a stopper 300, having similar or different volumes, to ease operators' need for accuracy in the first, sequestering drawing position. In this embodiment of the invention, stopper 300 can be formed with evacuated sequestration chambers 320 to enable fluids to be thereby drawn into said sequestration chambers.

### Methods

In an embodiment of the invention shown in FIG. 1A, sub-atmospheric pressure is created within sequestration chambers 120 of stoppers 100 as part of a process to form said sequestration chambers 120, sub-atmospheric pressure thus created within sequestration chamber 120 causes a first portion of a collection specimen to be drawn through proximal end 140 of needle 135 into said sequestration chamber 120.

Following collection and retention of a first portion of a specimen within sequestration chamber 120, proximal end 140 of needle 135 is pushed through bottom 106 of the stopper and subsequent portion of specimen deposited directly into collection device 150.

After collection of test specimen, a stopper can be discarded and a specimen of reduced contamination retrieved from collection chamber. Alternatively, as shown in FIG. 1B, a specimen of reduced contamination can be retrieved by a second, sampling needle 136 through a puncture of stopper 100 that avoids entering sequestration chamber 120.

The volume, shape, number and location of sequestration chambers 120 within stoppers 100 of this invention can be easily determined and adapted to specific needs by anyone of ordinary skill in the art.

In another embodiment of the invention, shown in FIG. 2A, proximal end 240 of a drawing needle 235 is first pushed into a sequestration chamber 220 in stopper 200 of a collection device such as a sample collection container 250.

As shown in FIG. 2A, sub-atmospheric pressure is created in sequestration chamber 220 through occluded opening 230, to a sub-atmospheric pressure within sample collection container 250, sub-atmospheric pressure thus created within sequestration chamber 220 causes the sequestration chamber to draw in a first portion of collection specimen. Specimen liquid then comes in contact with occlusions 231 within openings 230 in sequestration chamber 220. Contact with specimen liquid causes said occlusions to become relatively impermeable to liquid, and thereby causes liquid drawn in sequestration chamber 220 to become sealed within said sequestration chamber 220.

Following collection and retention of a first portion of a specimen within sequestration chamber 220, proximal end 240 of needle 235 is pushed through bottom 206 of a stopper and a subsequent portion of specimen deposited directly into space 255 of sample collection container 250.

After collection of a test specimen, the stopper can be discarded and a specimen of reduced contamination retrieved from space 255 in collection chamber 250. Alternatively, as shown in FIG. 2B, a specimen of reduced contamination can be retrieved through needle 236 by a second needle puncture through a sidewall 215 or 216 of a stopper that avoids entering sequestration chamber 220.

The volume, shape, number and location of sequestration chamber 220 within stoppers of this invention can be easily determined and adapted to specific needs by anyone of ordinary skill in the art.

Type, material, functional pore size and uniform or non-uniform hydrophobicity of material to be loaded in openings 230 between sequestration chamber 220 and space 255 in sample collection chamber 250 can be easily determined and adapted to specific needs by anyone of ordinary skill in the art.

In yet another embodiment of the invention, shown in FIG. 3A, sub-atmospheric pressure is created within a set of stacked sequestration chambers 320 and 321 of a stopper as part of process of their manufacture. Sub-atmospheric pressure thus created within sequestration chambers 320 and 321 cause first portion of a collection specimen to be drawn into said sequestration chamber 320.

Following collection and retention of a first and second portion of a specimen within sequestration chamber 320 and 321, respectively, tip end 340 of a needle 335 is pushed through bottom 306 and subsequent portion of specimen deposited directly into space 355 of sample collection container 350.

After collection of test specimen, the stopper can be discarded and uncontaminated specimen retrieved from collection chamber 350. Alternatively, as shown in FIG. 3D, specimen with reduced contamination can be retrieved through sample collection needle 336 inserted through the sidewall 315 or 316 of the stopper in a path that avoids entering sequestration chamber 320 or 321.

The volume, shape, number and location of sequestration chambers 320 and 321 within stoppers of this invention can be easily determined and adapted to specific needs by anyone of ordinary skill in the art.

### EXAMPLES

The following examples are intended to illustrate aspects of this invention, but are not intended to limit the scope of the invention. Persons of skill in the art can readily create other embodiments based on the disclosures and teachings herein without undue experimentation and with a reasonable likelihood of success. All such embodiments are considered to be part of this invention.

### Example 1

### Device to Sequester a First Amount of Fluid I

FIG. 1A depicts a device 100 of this invention to sequester a first amount of blood or other fluid drawn from a patient into a sub-atmospheric collection device such as a sample collection container 150. The device comprises a stopper having a top portion 105, sidewalls 115 and 116, and bottom portion 106, defining sequestration chamber 120 therein. Sequestration chamber 120 is sized to hold a first amount of blood or other bodily fluid and is capable of holding from 1 cubic millimeter to 10,000 cubic millimeters or more of blood or other fluid. Top 105 has flange 110 to prevent the stopper from being drawn into the sample collection container 150 by sub-atmospheric pressure in the collection container 150.

A stopper can be formed of resilient rubber or other similar material.

Sequestration chamber 120 within stopper 100 is formed having sub-atmospheric pressure therein, that enables said sequestration chamber 120 to draw a first portion of blood or other bodily fluid into sequestration chamber 120.

As shown in FIG. 1B, stopper 100 is formed with sidewalls 115 and 116 of sufficient thickness to enable a drawing needle 136 to traverse the stopper without touching or going through any sequestration chamber 120. Safe passage of the needle through the stopper is ensured by use of needle-puncturable material in formation of any part of the stopper supporting access of needle 136.

### Example 2

### Device to Sequester a First Amount of Fluid II

FIG. 2A depicts a device of this invention to sequester a first amount of blood or other fluid drawn from a patient into a sub-atmospheric collection device such as a sample collection container 250. The device comprises a stopper containing a sequestration chamber 220 to hold a first amount of a test sample of blood or other bodily fluids. Sequestration chambers 220 is capable of holding from 1 cubic millimeter to 10,000 cubic millimeters or more of blood or other fluid. The stopper has flange 210 at its top to prevent said the stopper from being drawn into the sample collection container by sub-atmospheric pressure of the sample collection container.

Sequestration chamber 220 is formed between top 205 and bottom 206, with sidewalls 215 and 216, with one or more windows 225 of rectangular, circular, oval or any other shape. Through said windows, an operator or phlebotomist can observe blood or fluid enter said sequestration chamber 220. Said windows are sealingly covered by a transparent wall of said sample collection container 250.

Sequestration chamber 220 has one or more openings 230 of circular, oval, or other form, extending from sequestration chamber 220 to space 255 of sample collection container 250. Openings 230 are filled with material 231 of such absorbency and porosity that when wetted, said material becomes wetted material 231a, and becomes relatively impermeable to gas and fluid, thus hindering flow of gas and blood or fluid through said openings 230.

Openings 230 within a stopper can be single, multiple circular, at periphery of stopper 200 or arranged in any suitable number in positions to guarantee that regardless of position of sample collection container during blood or fluid drawing, some openings 230 will remain dry and therefore permeable to gas until said sequestration chamber 220 is sufficiently filled with fluid.

As shown in FIG. 2B, a stopper is formed so as to enable a sample collection needle 236 to transverse the sidewalls 215 or 216 of said stopper without touching or going through sequestration chamber 220. Safe passage of sample collection needle 236 through the stopper is ensured by use of needle-puncturable material in formation of sidewalls 215 and 216.

### Example 3

### Device to Sequester First Amount of Fluid III

FIG. 3A depicts a device of this invention to sequester a first amount of blood or other fluid drawn from a subject into a sub-atmospheric collection device such as a sample collection container 350. The device comprises a stopper containing sequestration chambers 320, and 321, each capable of holding from 1 cubic millimeter to 10,000 cubic millimeters or more of blood or other fluid. The stopper has flange 310 at its top to prevent the stopper from being drawn into the sample collection container 350 by sub-atmospheric pressure in the space 355 of collection container 350.

Sequestration chambers 320 and 321 are formed between top 305 and bottom 306 of the stopper separated by sidewalls 315 and 316 constructed of resilient rubber or other similar material, with one or more windows of rectangular, circular, oval or any other shape between said parts of the stopper. Through said windows, an operator or phlebotomist can observe blood or fluid enter said sequestration chambers 320 and 321. The windows are sealingly covered by a transparent wall of sample collection container 350.

Stacked sequestration chambers 320 and 321 with similar or different sequestration volumes is provided to ease an operator's need for accuracy in the first, sequestering drawing position. Sequestration chambers 320 and 321 are formed with sub-atmospheric pressure therein. The stopper is formed so as to leave a continuous solid area between lower and upper parts of the stopper through which said area a sample collection needle 336 can transverse said stopper without touching or going through sequestration chambers 320 or 321. Safe passage of needle through the stopper is ensured by use of needle-puncturable material in formation of sidewalls 315 or 316 of said stopper,

### Example 4

### Method to Sequester First Amount of Fluid I

Other aspects of this invention include methods where the blood or fluid sequestering function of a stopper of Example 1. In FIG. 1A, a portion of a distal needle (not shown) is placed in a vein, artery or other source of fluid. Fluid is then drawn through the distal end of the needle to a proximal needle 135, which is inserted through top 105 of the stopper, with the end tip 140 of said proximal needle penetrating into sequestration chamber 120.

Sub-atmospheric pressure raging from about one (1%) to about 90% of the surrounding atmospheric pressure within sequestration chamber 120 causes a first portion of blood or other fluid of a test sample to be drawn into said sequestration chamber 120.

When the sequestration chamber 120 fills up to a desired level with a first portion of drawn blood or bodily fluid, an operator pushes the tip end 140 further, of proximal needle 135 through bottom 106, so that said tip end 140 of proximal needle 135 protrudes into sample collection container 155.

A first amount of blood or fluid drawn, that is often contaminated by a skin plug, skin cells, bacteria, fungi, viruses and their respective RNA or DNA, or specific molecules or disinfectant is thus sequestered in sequestration chamber 120. As shown in FIG. 1B, an uncontaminated sample can be drawn out through a needle 136 which does not transverse said sequestration cavities 120.

### Example 5

### Method to Sequester First Amount of Fluid II

In this example, a distal needle is placed in a vein, artery or other fluid-containing cavity. Fluid flows through the distal needle and into a proximal needle inserted into a stopper of a sample collection container 250, with tip end 240 of said needle 235 penetrating into the sequestration chamber 220.

Sub-atmospheric pressure in the sample collection container is equilibrated with the pressure in sequestration chamber 220 through opening 230. Absorbent, occlusion-creating material 231 present in opening 230 is wetted by the fluid. Fluid wets the material 231 which expands, thereby producing wetted material 231a, expanding and filling occluding opening 230. Opening 230 becomes relatively impermeable to air, gas, blood or fluid when filled with wetted material 231a. Fluid flow between sequestration chamber 220 and the space 255 of collection container 250 stops when sequestration chamber 220 becomes filled and all absorbent filled openings 230 wetted and no longer permeable to air or gas to transmit_the sub-atmospheric drawing pressure.

When operator observes a sequestration chamber 220 within a stopper 200 fill up to a desired level with the first portion of the drawn blood or bodily fluid, the operator pushes the proximal end 240 of proximal needle 235. Further, through bottom 206, so that said tip 240 of proximal needle 235 then protrudes and opens into space 255 of sample collection container 250.

A first amount of blood or fluid drawn, that is often contaminated by a skin plug, skin cells, bacteria, fungi, viruses and their respective RNA, DNA, or specific molecules or disinfectant is thus sequestered in said sequestration chamber. An uncontaminated sample can then be drawn out through sample collection needle 236, in a location on the stopper assembly as described in Example 3, so that it does not transverse said sequestration chamber 220.

### Example 6

### Method to Sequester First Amount of Fluid III

In this example, a distal needle is placed in a vein, artery or natural or pathological space or other fluid-filled cavity. Fluid flows through the distal needle to a proximal needle 335 that is inserted into a stopper of a sample collection container 350, with tip end 340 of said proximal needle 335 penetrating into selected stopper sequestration chamber 320.

Sub-atmospheric pressure ranging from 1% to 90% of surrounding atmospheric pressure within sequestration chambers 320 causes a first portion of blood or other fluid of a test sample to be drawn into selected sequestration chamber 320. The fluid drawn into sequestration chamber 320 is visible to operator or phlebotomist through a transparent wall of the sample collection container.

When an operator observes a sequestration chamber 320 to be sufficiently filled by a first portion of the drawn blood or bodily fluid, the operator pushes the tip end 340 of proximal needle 335 further, through bottom 306, so that said tip end 340 protrudes into space 355 of sample collection chamber 350.

A first amount of blood or fluid drawn, that is often contaminated by a skin plug, skin cells, bacteria, fungi, viruses and their respective RNA, DNA, or specific molecules or disinfectant is thus sequestered in chamber 320. As shown in FIG. 3B, an uncontaminated sample can then be drawn out of space 355 in sample collection container 350 through sample collection needle 336 for subsequent analysis.

### Example 7

### Method to Sequester First Amount of Fluid IV

In this example, threaded attachment of a stopper to a collection container as described in FIGS. 4A-4D. A distal needle (not shown) is placed in a vein, artery, cyst, or other fluid-containing portion of a subject's body. Proximal needle 435, attached to the distal needle by way of a tube, and in fluid communication with the distal needle is inserted through top 405 of a stopper and the tip end 440 of the proximal needle 435 is placed within sequestration chamber 420. The first portion of the collected sample is drawn into the sequestration chamber due to the sub-atmospheric pressure therein, and remains sequestered there. Subsequently, the tip end 440 of the proximal needle 435 is inserted through the bottom 406 and into space 455 of a sample collection container 450, where a relatively uncontaminated sample is drawn by sub-atmospheric pressure and is deposited within space 455. Subsequently, a sample collection needle 436 is inserted through sidewall 415 or 416 and into space 455 containing the relatively uncontaminated sample. A sample is thereby withdrawn for testing, storage, or other purpose.

### Example 8

### Method to Sequester First Amount of Fluid V

In this example a device as shown in FIGS. 5A-5D is used. In this example, a distal needle (not shown) is placed in a vein, artery, cyst, or other fluid-containing portion of a subject's body. A proximal needle 535, attached to the distal needle by way of a tube, and in fluid communication with the distal needle is inserted through top 505 and the tip end 540 is placed within sequestration chamber 520. The first portion of the collected sample is drawn into the sequestration chamber due to the sub-atmospheric pressure therein. In this example an opening 530 exists through the bottom 506 of the sequestration chamber 520. An absorbent, occlusion-creating material 531 is within opening 530, and when the first portion of fluid is deposited in the sequestration chamber, a portion of the fluid is absorbed by the absorbent material, thereby forming wetted material 531a, causing it to swell and occlude the opening 530 through bottom 506 of the sequestration chamber 520. Therefore, a first portion of the fluid sample remains sequestered there.

Subsequently, tip end 540 of the proximal needle 535 is inserted through bottom 506 of sequestration chamber 520 and into a space 555 within a collection container 550, where a relatively uncontaminated sample is drawn by sub-atmospheric pressure and is deposited within space 555. Subsequently a sampling needle 536 is inserted through a sidewall 515 or 516 and into space 555 containing the relatively uncontaminated sample. A sample is thereby withdrawn for testing, storage, or other purpose.

### Example 9

### Method to Sequester a First Amount of Fluid VI

In another aspect, devices may contain two sequestration chambers 620 and 621 as shown in FIGS. 6A-6B. As proximal needle 635 is passed successively through the sequestration chambers 620 and 621, additional portions of the fluid sample may be sequestered, and therefore not be deposited into space 655 of collection container 650.

### Example 10

### Method to Sequester First Amount of Fluid VII

An “in-line” embodiment of the invention is used to collect a sample of culture fluid from a culture bag. Such devices are described above with reference to FIGS. 7A 7B, 8A-8D, and FIGS. 9A and 9B. In these embodiments, a sequester chamber 720, 820 or 920 is not connected to a collection container, but rather, is used in conjunction with a collection tube, which may be used to further transport culture fluid through tube 755, 855 or 955 into another location, including a sample collection container (not shown).

In FIG. 9, culture bag 902 may contain growing cells, growth medium (fluid), and/or other components of the culture. Cells and tissues in the culture bag 902 may produce materials of interest, including proteins, metabolites, nucleic acids and other materials. It is often desirable to analyze the materials within the culture bag. To avoid contaminating such samples with bacteria, viruses, and other undesirable materials present on the outside of the bag, an in-line device of this invention is used to penetrate a portal 910 on the culture bag 902, then to transport the first portion of the sample into sequestration chamber 920 at sub-atmospheric pressure, where the first portion (containing contaminants) is deposited and sequestered. Subsequently proximal needle 935 is inserted through right side 906 of sequestration chamber 920 and into a sample collection tube 955. Thus, the material in the sampling tube 955 is relatively free of contaminants.

The aforementioned examples are by way of illustration only, and not intended to limit the scope of this invention. Other embodiments based on the disclosure and teachings can be used by persons of skill in the art and all such embodiments are considered part of this invention.

Each of the examples described herein can be used either singly or in combination, as desired. The described invention represents a significant improvement over the prior art devices, and can be used to produce a great technical improvement and excellent effect in sampling fluids for analysis. The inventions described herein solve a substantial, major unmet need in the art, and produce highly unexpected results, based on those of the prior art. Therefore, the inventions as described are novel, not obvious, and are highly inventive.

1. A device, comprising: a stopper having a bottom portion, a sidewall portion, and a top portion defining a sequestration chamber impermeable to air, having sub-atmospheric pressure therein; said top, sidewall, and bottom portions being penetrable by a needle.

2. The device of claim 1, further comprising a sample container having a closed end and an open end, said sample container sized to sealingly accept said stopper.

3. The device of claim 1, said stopper comprising: a screw cap having a bottom portion, a sidewall portion, and a top portion defining a sequestration chamber impermeable to air, said sequestration chamber having sub-atmospheric pressure therein, said screw cap threadably engaged with corresponding threads on said sample container, said screw cap penetrable by a needle; and a sample collection container having a closed end and an open end, said sample collection container sized to threadably and sealingly engage said screw cap.

4. The device of claim 1, where said sequestration chamber has a volume ranging from about 1 cubic millimeter to about 10,000 cubic millimeters.

5. The device of claim 1, further comprising one or more additional sequestration chambers containing sub-atmospheric pressure.

6. The device of claim 1, where said sample container has sub-atmospheric pressure therein.

7. The device of any of claim 1, where the pressure within at least one of said sequestration chambers is in the range of about 1% to about 90% of the surrounding atmospheric pressure.

8. The device of claim 1, at least one of said walls being transparent.

9. The device of claim 5, said sequestration chambers varying in progressive fashion from larger to smaller in a distal direction.

10. The device of claim 5, said sequestration chambers varying in progressive fashion from smaller to larger in a distal direction.

11. The device of claim 1 said sequestration chamber sized to hold a volume of fluid from about 1 cubic millimeter to about 10,000 cubic millimeters.

12. The device of claim 1, said sequestration chamber comprising a window to permit an observer to see within the sequestration chamber or chambers.

13. The device of claim 1, where a top portion of the stopper comprises a flange extending above the top of an open end of the sample collection container.

14. The device of claim 1, where the bottom portion of at least one sequestration chamber has an opening extending from the sequestration chamber through the bottom portion of the stopper or screw cap, the opening further containing a porous, absorbent material that when wetted by a fluid, occludes the openings and thereby prevents gas or fluid from passing from the sequestration chamber into the sample collection container.

15. The device of claim 14, where said absorbent material is selected from the group consisting of packed or woven fibers of cotton, cellulose, cellulose fibers, polyamines, cationic starch, rayon, cotton, silk, nylon, microporous polyvinylidene difluoride (PVDF), microporous mixed cellulose esters (MCE), and poly tetra fluoro ethylene (PTFE), mixed cellulose esters (MCE), acrylonitrile butadiene styrene (ABS), butadiene styrene rubber (BS), cyclohexanedimethanol (CHDM), cellulose nitrate-cellulose acetate (CN-CA), ethylene propylene terpolymer rubber (EPDM), ethylene vinyl acetate (EVA), high density polyethylene (HDPE), high density polypropylene (HDPP), high impact polystyrene (HIPS), low density polyethylene (LDPE), methyl methacrylate ABS (MABS), nitrile butadiene rubber (NBR), neoprene (NPRN), polyamide (PA), polybutylene terephthalate (PBT), polycarbonate (PC), polyethylene (PE), polyethersulfone (PES), polyethylene terephthalate (PET), PET modified with glycolor (PETG), polyimide (PI), butyl rubber (PIB), polyoxymethylene (POM), polypropylene (PP), polystyrene (PS), polysulfone (PSU), polytetrafluoroethylene (PTFE), polyurethane (PU), polyvinyl acetate (PVA), polyvinyl chloride (PVC), styrene butadiene (SB), styrene butadiene rubber (SBR), stainless steel (SS), thermoplastic elastomer (TPE), thermoplastic rubber (TPR).

16. The device of claim 15, where said absorbent material has an effective pore size ranging from 0.02 μm to 1 mm.

17. (canceled)

18. A device for collecting a sample of fluid from a culture container, comprising:
a first side, a second side, a third side, a fourth side, a top, and a bottom, defining a first sequestration chamber therebetween, said first sequestration chamber having sub-atmospheric pressure therein, said top and bottom being penetrable by a needle.

19. The device of claim 18, further comprising a second sequestration chamber having sub-atmospheric pressure therein, and separated from said first sequestration chamber by the bottom of said first sequestration chamber.

20. A method for collecting a sample of fluid from a subject, comprising;
a. providing a device of claim 1;
b. providing a sampling device having a distal needle and a proximal needle connected to each other by a tube defining a continuous fluid passageway;
c. inserting the distal needle into a fluid-filled cavity of a subject, permitting said fluid to flow through said distal needle, said tube, and into the proximal needle;
d. inserting the proximal needle through the top portion of said stopper and into said sequestration chamber, permitting a first portion of the fluid to be drawn into the sequestration chamber; then
e. inserting the proximal needle through said bottom portion of said sequestration chamber and into a sample container, thereby permitting a second portion of fluid to flow into the sample container.

21-25. (canceled)
